(19) United States
(12) Patent Application Publication
(10) Pub. No.: US 2003/0166589 A1
(43) Pub. Date: Sep. 4, 2003

(54) Method and pharmaceutical composition for the treatment of multiple sclerosis

(76) Inventor: Nathan Karin, Haifa (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(21) Appl. No.: 09/873,415

(22) Filed: Jun. 5, 2001

(51) Int. Cl. A61K 48/00; A61K 39/00

(52) U.S. Cl. 514/44; 424/185.1

(57) ABSTRACT

Methods and pharmaceutical compositions are disclosed, effective in breaking-down immunological tolerance the CXC chemokine interferon gamma-inducible protein 10 (IP-10), resulting in the generation of self specific immunity to IP-10, for the treatment of diseases, such as autoimmune diseases, in which IP-10 plays a pivotal role in disease onset and/or progression, e.g., in multiple sclerosis (MS).

### FIELD AND BACKGROUND OF THE INVENTION

The present invention relates to methods and pharmaceutical compositions effective in breaking-down immunological tolerance the CXC chemokine interferon gamma-inducible protein 10 (IP-10), resulting in the generation of self specific immunity to IP-10, for the treatment of diseases, such as autoimmune diseases, in which IP-10 plays a pivotal role in disease onset and/or progression, e.g., multiple sclerosis (MS) and other inflammatory autoimmune diseases such as rheumatoid arthritis. Thus, in one particular, the present invention relates to the induction of protective immunity against multiple sclerosis, so as to prevent or treat multiple sclerosis by DNA vaccines or by neutralizing antibodies directed to IP-10.

Experimental autoimmune encephalomyelitis (EAE) is an autoimmune disease of the central nervous system (CNS) which, for many years and for a variety of experimental protocols, serves as a model for the human disease, multiple sclerosis (MS), a chronic degenerative disease marked by patchy destruction of the myelin that surrounds and insulates nerve fibers and mild to severe neural and muscular impairments, since in both diseases circulating leukocytes penetrate the blood brain barrier and damage myelin resulting in impaired nerve conduction and paralysis (1, 2).

Molecular biology techniques were previously used to follow leukocyte trafficking to the site of inflammation at the CNS of EAE rats, and a model that characterizes this process as a sequential multi-step event was suggested (3).

At first, a very limited repertoire of T-cells, named “the primary influx” interact with their target antigen at the site of inflammation, leading to the activation of the blood brain barrier to express various adhesion molecules and thus to increase its permeability to circulating leukocytes (3, 4). Enhanced permeability of this barrier allows a non-selective influx of leukocytes, which are named “the secondary influx”. This influx correlates with disease onset (3, 5). Subsequently, antigen specific autoimmune T-cells either become anergic or undergo programmed cell death (apoptosis) leading to a remission in disease severity (6). Inhibition of the secondary influx, by either soluble peptide therapy or anti-adhesion molecule blockade effectively prevented, or even reversed, an ongoing disease even though the primary influx remained apparent at the site of inflammation (3-5, 7). Taken together these results emphasize the important role of the non-selective leukocyte influx to a site of inflammation.

During the course of EAE development, various proinflammatory cytokines and chemokines are produced at the site of inflammation (40, 53-55).

Chemokines are chemoattractants that mediate leukocyte attraction and recruitment at the site of inflammation. As such, they are likely to be key mediators in the recruitment of the secondary influx of leukocytes at an inflamed target organ. This has motivated researchers to use the novel technology of naked DNA vaccination (8-17) and explore the therapeutic potential of anti-chemokine immunotherapy in EAE.

Based on the positions of the first two cysteines, the chemokines can be divided into four highly conserved but distinct supergene families C-C, C-X-C, C and the newly discovered C-X3-C (18, 19, 36-38). The C-C family is primarily involved in the activation of endothelium and for chemoattraction of T cells and monocytes to the site of inflammation (20-32).

The protective competence of anti-C-C chemokine based immunotherapy has been demonstrated by Karpus at al. who blocked EAE in mice by immunizing them with rabbit anti-mouse polyclonal antibodies against macrophage inflammatory protein-la (MIP-1α (33), and more recently by Gong at al. who used an antagonist of monocyte chemoattractant protein 1 (MCP-1) to inhibit arthritis in the MRL-1pr mouse model (34). In another study, Berman at al. used in situ hybridization to demonstrate the dominant expression of MCP-1 in rat EAE brain (35). Still more recently, Barnes et al. used anti-human RANTES antibody to ameliorate adjuvant induced arthritis in the Lewis rat (87).

The pivotal role of the proinflammatory cytokine tumor necrosis factor alpha (TNF-α) in EAE has also been well characterized. TNF-α is produced by activated T cells (mostly Th1) and macrophages, and its elevated expression at the site of inflammation occurs during the critical phase of disease (55), at the time when the ‘secondary influx’ of leukocytes is apparent (3). Except for a single recent study carried out in genetically modified animals (56), all investigators agree that TNF-α contributes to the proinflammatory process in EAE and MS (57-71). Early studies have shown that IFN-γ and TNF-α together exhibit a synergistic effect on enhancing expression of adhesion molecules on endothelial cells (61), and on eliciting the inflammatory process, which can be reversed by either anti-adhesion molecule immunotherapy (4), or by blocking TNF-α (57-61). More recent studies have demonstrated that inhibition of TNF-α activity by either neutralizing antibodies, or soluble TNF receptor therapy, effectively prevent, or even reverse EAE (62, 64, 66-71). Overexpression of TNF-α at the CNS aggravated the disease (65), whereas genetically impaired expression of this gene inhibited disease development and progression (63).

A major disadvantage in treating chronic diseases with xenogenic neutralizing antibodies lies in their immunogenicity. This has motivated investigators to develop chimeric humanized antibodies (reviewed in 50), and monoclonal antibodies engineered with human Ig heavy and light chain yeast artificial chromosome (YAC) (51). However, following repeated immunization, these engineered antibodies do trigger an apparently allotypic response.

DNA vaccines represent a novel means of expressing antigens in vivo for the generation of both humoral and cellular immune responses (10, 14, 39, 41-43). This technology has proven successful in obtaining immunity not only to foreign antigens and tumors, but also to self antigens, such as a T cell receptor V genes (17) or autologous cytokines (42).

Recently, an alternative approach to generate anti chemokine/cytokine protective immunity was developed. WO 00/06203 teaches the use naked DNA vaccines expressing under the control of a viral promoter the C-C chemokines MCP-1, MIP-1α, macrophage inflammatory protein-1β (MIP-1β), regulation on activation normal T expressed and secreted (RANTES) or the cytokine tumor necrosis factor alpha (TNF-α) and a repeated immunostimulatory sequence (i.e., CpG motif) that serves as a DNA adjuvant (15, 39, 88, 89), in the induction of protective immunity against multiple sclerosis.

Repeated administrations of such self cytokines encoding DNA vaccines tolerance to each relevant gene product is broken and immunological memory is established (90-96). These studies have also demonstrated that following the initiation of a T cell mediated autoimmune condition this memory is “turned on” to provide protective immunity (90-96). Thus, administration of either TNF-α, MIP-1α or MCP-1 DNA vaccines rendered high state of resistance against two different autoimmune diseases EAE and adjuvant induced arthritis (AA) that could be adoptively transferred by the neutralizing antibodies generated in response to each gene product. C-C chemokines were selected as candidates for DNA vaccination mostly because of their well established role in cell migration to a target organ (22, 23, 44-49). Since DNA vaccination elicits both cellular and humoral responses against products of a given construct (10, 14, 41-43), it is difficult to know which of these responses contributed more to the development of resistance. Nevertheless, elicitation of these antibodies was found to be dependent on the development of an autoimmune condition and regulated by the immune system in accordance with disease progression (90, 94). This, however, could provide the immune system of a patient with an autoimmune condition a powerful tool with which it can restrain its own harmful activities (94). Hence, a point of concern is that as an adjuvant the CpG may select Th1 cells that may potentially aggravate autoimmunity.

The role of the CXC chemokine interferon gamma-inducible protein 10 (IP-10) in the regulation of EAE or MS is yet unknown. Recent studies demonstrate IP-10's ability to stimulates the directional migration of activated T cells, particularly Th1 cells (97-99), including of human T cell in SCID mice (100). Other studies demonstrate that IP-10 is transcribed at the CNS of MS patients, and NOD mice with developing type I diabetes (101-104).

While conceiving the present invention it was hypothesized that if IP-10 has a pivotal role in the development and/or progression of EAE or MS, than, it cab be used for the induction of protective immunity against multiple sclerosis, so as to prevent or treat multiple sclerosis. One advantage of using IP-10 for the induction of protective immunity lies in that downregulation of IP-10 via naked DNA vaccination would also reduce the chances of directional migration of activated T cells, particularly Th1 cells, that may potentially aggravate autoimmunity and which may be selected via the CpG motif adjuvant activity.

### SUMMARY OF THE INVENTION

Interferon gamma-inducible protein 10 (IP-10) is a CXC chemokine that stimulates the directional migration of activated T cells, particularly Th1 cells. While reducing the present invention to practice, it was found that administration of plasmid DNA encoding self IP-10 was efficient in breaking down immunological tolerance to IP-10, resulting in the generation of self specific immunity to IP-10. Despite the fact that the plasmid vaccine contained a repeated immunostimulatory sequence (i.e., a CpG motif) that serves as a DNA adjuvant and that is known to redirect Th1 polarization, the vaccine redirected the polarization of myelin basic protein specific T cells into Th2 and conferred the vaccinated rats a high state of resistance against experimental autoimmune encephalomyelitis (EAE), a T cell mediated autoimmune disease of the central nervous system (CNS) which traditionally serve as an in animal model system of multiple sclerosis (MS). The vaccine also suppressed disease when being administered after its active induction. Self specific antibodies to IP-10 developed in protected animals could inhibit leukocyte migration in a Boyden chamber, alter the in vivo Th1/Th2 balance of autoimmune T cells, and adoptively transfer disease suppression. EAE resistance was associated with an apparent alteration in the in vivo Th1/Th2 balance of autoimmune T cells towards Th2.

Thus, according to one aspect of the present invention there is provided a method of breaking-down an immunological tolerance to interferon gamma-inducible protein 10 in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to break-down the immunological tolerance to interferon gamma-inducible protein 10.

According to another aspect of the present invention there is provided a method of breaking-down an immunological tolerance to interferon gamma-inducible protein 10 in a subject, the method comprising administering to the subject anti-interferon gamma-inducible protein 10 antibodies in an amount sufficient to break-down the immunological tolerance to interferon gamma-inducible protein 10.

According to still another aspect of the present invention there is provided a method of breaking-down an immunological tolerance to interferon gamma-inducible protein 10 in a subject, the method comprising directly or indirectly introducing anti-interferon gamma-inducible protein 10 antibodies to the subject in an amount sufficient to break-down the immunological tolerance to interferon gamma-inducible protein 10.

According to yet another aspect of the present invention there is provided a pharmaceutical composition for breaking-down an immunological tolerance to interferon gamma-inducible protein 10 in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding the interferon gamma-inducible protein 10, or the immunological portion thereof, sufficient to elicit sufficient anti-interferon gamma-inducible protein 10 antibodies so as to break-down the immunological tolerance to interferon gamma-inducible protein 10.

According to an additional aspect of the present invention there is provided a pharmaceutical composition for breaking-down an immunological tolerance to interferon gamma-inducible protein 10 in a subject, the pharmaceutical composition comprising, a pharmaceutically acceptable carrier approved for medical or veterinary administration and, as an active ingredient, anti-interferon gamma-inducible protein 10 antibodies in an amount sufficient to break-down the immunological tolerance to interferon gamma-inducible protein 10.

According to yet another aspect of the present invention there is provided a method of generating self specific immunity to interferon gamma-inducible protein 10 in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to generate self specific immunity to interferon gamma-inducible protein 10.

According to a further aspect of the present invention there is provided a method of generating specific immunity to interferon gamma-inducible protein 10 in a subject, the method comprising administering to the subject anti-interferon gamma-inducible protein 10 antibodies in an amount sufficient to generate self specific immunity to interferon gamma-inducible protein 10.

According to still a further aspect of the present invention there is provided a method of generating specific immunity to interferon gamma-inducible protein 10 in a subject, the method comprising directly or indirectly introducing anti-interferon gamma-inducible protein 10 antibodies to the subject in an amount sufficient to generate self specific immunity to interferon gamma-inducible protein 10.

According to yet a further aspect of the present invention there is provided a pharmaceutical composition for generating self specific immunity to interferon gamma-inducible protein 10 in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding the interferon gamma-inducible protein 10, or the immunological portion thereof, sufficient to elicit sufficient anti-interferon gamma-inducible protein 10 antibodies so as to generate self specific immunity to interferon gamma-inducible protein 10.

According to another aspect of the present invention there is provided a pharmaceutical composition for generating self specific immunity to interferon gamma-inducible protein 10 in a subject, the pharmaceutical composition comprising, a pharmaceutically acceptable carrier approved for medical or veterinary administration and, as an active ingredient, anti-interferon gamma-inducible protein 10 antibodies in an amount sufficient to generate self specific immunity to interferon gamma-inducible protein 10.

According to an additional aspect of the present invention there is provided a method of preventing or treating an autoimmune disease in which activity of interferon gamma-inducible protein 10 is pivotal in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to treat or prevent the autoimmune disease.

According to still an additional aspect of the present invention there is provided a method of preventing or treating an autoimmune disease in which activity of interferon gamma-inducible protein 10 is pivotal in a subject, the method comprising administering to the subject anti-interferon gamma-inducible protein 10 antibodies in an amount sufficient to treat or prevent the autoimmune disease.

According to yet another aspect of the present invention there is provided a method of preventing or treating an autoimmune disease in which activity of interferon gamma-inducible protein 10 is pivotal in a subject, the method comprising directly or indirectly introducing anti-interferon gamma-inducible protein 10 antibodies to the subject in an amount sufficient to treat or prevent the autoimmune disease.

According to yet an additional aspect of the present invention there is provided a pharmaceutical composition for preventing or treating an autoimmune disease in which activity of interferon gamma-inducible protein 10 is pivotal in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding the interferon gamma-inducible protein 10, or the immunological portion thereof, sufficient to elicit sufficient anti-interferon gamma-inducible protein 10 antibodies so as to treat or prevent the autoimmune disease.

According to sill another aspect of the present invention there is provided a pharmaceutical composition for preventing or treating an autoimmune disease in which activity of interferon gamma-inducible protein 10 is pivotal in a subject, the pharmaceutical composition comprising, a pharmaceutically acceptable carrier approved for medical or veterinary administration and, as an active ingredient, anti-interferon gamma-inducible protein 10 antibodies in an amount sufficient to treat or prevent the autoimmune disease.

According to a further aspect of the present invention there is provided a method of restricting a polarization of myelin basic protein specific T cells into Th2 cells in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to restrict a polarization of myelin basic protein specific T cells into Th2 cells.

According to still a further aspect of the present invention there is provided a method of restricting a polarization of myelin basic protein specific T cells into Th2 cells in a subject, the method comprising administering to the subject anti-interferon gamma-inducible protein 10 antibodies in an amount sufficient to restrict a polarization of myelin basic protein specific T cells into Th2 cells.

According to yet a further aspect of the present invention there is provided a method of restricting a polarization of myelin basic protein specific T cells into Th2 cells in a subject, the method comprising directly or indirectly introducing anti-interferon gamma-inducible protein 10 antibodies to the subject in an amount sufficient to restrict a polarization of myelin basic protein specific T cells into Th2 cells.

According to a further aspect of the present invention there is provided a pharmaceutical composition for restricting a polarization of myelin basic protein specific T cells into Th2 cells in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding the interferon gamma-inducible protein 10, or the immunological portion thereof, sufficient to elicit sufficient anti-interferon gamma-inducible protein 10 antibodies so as to restrict a polarization of myelin basic protein specific T cells into Th2 cells.

According to still another aspect of the present invention there is provided a pharmaceutical composition for restricting a polarization of myelin basic protein specific T cells into Th2 cells in a subject, the pharmaceutical composition comprising, a pharmaceutically acceptable carrier approved for medical or veterinary administration and, as an active ingredient, anti-interferon gamma-inducible protein 10 antibodies in an amount sufficient to restrict a polarization of myelin basic protein specific T cells into Th2 cells.

According to yet a further aspect of the present invention there is provided a method of inducing protective immunity against multiple sclerosis in a subject, the method comprising administering to, or expressing within, the subject an amount of interferon gamma-inducible protein 10, or an immunological portion thereof, sufficient to elicit sufficient anti-interferon gamma-inducible protein 10 antibodies, so as to induce protective immunity against multiple sclerosis in the subject.

According to another aspect of the present invention there is provided a method of inducing protective immunity against multiple sclerosis in a subject, the method comprising administering to the subject anti-interferon gamma-inducible protein 10 antibodies in an amount sufficient to induce protective immunity against multiple sclerosis in the subject.

According to still another aspect of the present invention there is provided a method of inducing protective immunity against multiple sclerosis in a subject, the method comprising directly or indirectly introducing anti-interferon gamma-inducible protein 10 antibodies to the subject in an amount sufficient to induce protective immunity against multiple sclerosis in the subject.

According to still a further aspect of the present invention there is provided a pharmaceutical composition for inducing protective immunity against multiple sclerosis in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding the interferon gamma-inducible protein 10, or the immunological portion thereof, sufficient to elicit sufficient anti-interferon gamma-inducible protein 10 antibodies, so as to induce protective immunity against multiple sclerosis in the subject.

According to yet another aspect of the present invention there is provided a pharmaceutical composition for inducing protective immunity against multiple sclerosis in a subject, the pharmaceutical composition comprising, a pharmaceutically acceptable carrier approved for medical or veterinary administration and, as an active ingredient, anti-interferon gamma-inducible protein 10 antibodies in an amount sufficient to induce protective immunity against multiple sclerosis in the subject.

According to another aspect of the present invention there is provided a method of preventing or treating multiple sclerosis in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to treat or prevent multiple sclerosis.

According to still another aspect of the present invention there is provided a method of preventing or treating multiple sclerosis in a subject, the method comprising administering to the subject anti-interferon gamma-inducible protein 10 antibodies in an amount sufficient to treat or prevent multiple sclerosis.

According to yet another aspect of the present invention there is provided a method of preventing or treating multiple sclerosis in a subject, the method comprising directly or indirectly introducing anti-interferon gamma-inducible protein 10 antibodies to the subject in an amount sufficient to treat or prevent multiple sclerosis.

According to a further aspect of the present invention there is provided a pharmaceutical composition for preventing or treating multiple sclerosis in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding the interferon gamma-inducible protein 10, or the immunological portion thereof, sufficient to elicit sufficient anti-interferon gamma-inducible protein 10 antibodies so as to treat or prevent multiple sclerosis.

According to still a further aspect of the present invention there is provided a pharmaceutical composition for preventing or treating multiple sclerosis in a subject, the pharmaceutical composition comprising, a pharmaceutically acceptable carrier approved for medical or veterinary administration and, as an active ingredient, anti-interferon gamma-inducible protein 10 antibodies in an amount sufficient to treat or prevent multiple sclerosis.

According to further features in preferred embodiments of the invention described below, expressing within the subject the amount of interferon gamma-inducible protein 10 is by generating in, or introducing into, the subject cells expressing recombinant interferon gamma-inducible protein 10, or an immunological portion thereof.

According to still further features in the described preferred embodiments generating in the subject cells expressing recombinant interferon gamma-inducible protein 10, or an immunological portion thereof, is by vaccinating the subject with an expression construct encoding interferon gamma-inducible protein 10, or the immunological portion thereof.

According to still further features in the described preferred embodiments the pharmaceutical composition is packaged and identified for treatment of a disease or condition in which interferon gamma-inducible protein 10 plays a pivotal role.

The present invention successfully addresses the shortcomings of the presently known configurations by providing novel methods and compositions with which to combat autoimmune diseases.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIGS. 1A-D demonstrate that IP-10 encoding DNA vaccine redirect antigen specific T cell polarization and suppresses EAE. A group of nine Lewis rats was subjected to four weekly injections of naked DNA encoding IP-10. Control rats (nine per group) were either injected with the pcDNA3 vector alone, or with PBS. Two months after the last immunization all rats were immunized with p68-86/CFA to induce active EAE. Just before the onset of disease (day 9) three rats per groups were sacrificed and spleen T cells were cultured together with MBPp68-86 for cytokine determination. The remaining six rats were monitored for the development of active EAE by an observer blind to the experimental protocol. FIG. 1A shows mean maximal score ±SE of these groups. FIGS. 1B-D show the levels of TNF-α, IL-4 and IFN-γ that was determined (mean triplicates±SE) in supernatants of spleen cells cultured as described above.

FIGS. 2A-C demonstrate that IP-10 encoding DNA vaccine induces breakdown of tolerance to its gene product and generates immunity to native IP-10. Groups of Lewis rats were subjected to a repeated administration of IP-10 encoding DNA vaccines and then to active induction of EAE as described in the legend to FIG. 1. An additional group was subjected to IP-10 encoding DNA vaccine and later on immunized with CFA alone to induce a local inflammatory response. At different time points blood serum was obtained and evaluated for IP-10 specific antibody titer. On day 12 (peak of disease in control rats) SCF was also obtained and subjected to the same evaluation. FIG. 2A shows by Western Blot demonstrating that the self specific anti IP-10 antibodies bind to recombinant rat IP-10 (10 kDa) and also to the commercially available mouse IP-10 (8.7 kDa fragment, Cytolab, Rehovot, Israel). These antibodies also bound natural rat IP-10 from supernatant of activated MBP p68-86 specific cultured T cells (not shown). FIG. 2B shows the IP-10 specific antibody titer developed in blood serum and SCF of representative rats from each of the above groups (Mean antibody titer obtained from 3 rats per group±SE). FIG. 2C shows the kinetics of antibody titer to self IP-10 in pcDNA3-IP vaccinated rats that were subjected to either active induction of EAE or administration of CFA alone (Mean antibody titer obtained from 3 rats per group±SE).

FIGS. 3A-C demonstrate that IP-10-specific antibodies generated in DNA vaccinated EAE rats are neutralizing antibodies that inhibit both migratory properties (FIG. 3A) and polarization of activated T cells, as was measured by direct ELISA (FIG. 3B) or by intracellular FACS analysis (FIG. 3C). In FIG. 3A, IP-10 specific antibodies (IgG, CNBr purified) were tested for their ability to inhibit the migration of an encephalitogenic cell line (L68-86) in a Boyden chemotaxis chamber assay. Rat IP-10 and the 8.7 kDa fraction of mouse IP-10 (Cytolab), each at a concentration of 200 ng/ml, were used as chemoattractants. fMLP (Sigma) at a concentration of 10⁻⁷ M was used as a positive control for chemoattraction. Result are shown as mean of triplicates ±SE. Boyden chemotaxis chamber assay. The experimental protocol for FIGS. 3B and 3C is as follows: Nine days after active EAE induction primary spleen T cells were cultured with 40 μg/ml of MBP p68-86 with or without, the addition of 10 ng/ml of either anti IP-10 antibodies, antibodies from control AE rats vaccinated with an empty plasmid, control IgG from naïve rats or medium. Production of TNF-α (FIG. 3A), IFN-γ (FIG. 3B) and IL-4 (FIG. 3C) were determined 72 h later. At that time CD4+ spleen T cells (W3/25+) from cultures that were or were not supplemented with anti IP-10 antibodies were subjected to intracellular FACS analysis of IFN-γ vs IL-4 (FIG. 3C).

FIGS. 4A-D demonstrate that administration of self specific antibodies to IP-10 redirects antigen specific T cell polarization towards Th2 and suppresses EAE. Four groups of nine rats were subjected to active induction of EAE. During the first five days following disease induction these rats were repeatedly (every other day) subjected to injections of 100 μg/rat of either anti IP-10 antibodies, IgG from pcDNA3 vaccinated EAE rats or from normal rat serum. Another control group was administered with PBS. Three rats per group were sacrificed on day 10 and their spleen T cells were cultured together with MBPp68-86 for cytokine determination (FIGS. 4B-D) and FACS analysis (FIG. 5). The remaining rats were daily monitored for clinical signs of disease by an observer blind to the experimental protocol. FIG. 4A shows mean maximal score±SE of these groups. FIGS. 4B-D show the levels of TNF-αIL-4 and IFN-γ that were determined (mean triplicates±SE) in supernatants of spleen cells derived from the rats and cultured as described above.

FIGS. 5A-B demonstrate intracellular staining of IL-4 and IFN-γ in primary spleen cell cultures of rats administered with anti-IP-10 specific antibodies. Cultured spleen cells (48 hours of in vivo stimulation) derived from EAE rats that were subjected to treatment with anti-IP-10 specific antibodies (FIG. 5A), PBS (FIG. 5B), or IgG from normal rat serum (not shown), were analyzed for intracellular IL-4 vs. IFN-γ of CD4+ (w3/25+) T cells. No difference were recorded between T cells from rats which received no treatment (FIG. 5A) to those from rats treated with normal rat IgG (not shown).

FIG. 6 demonstrate that IP-10 encoding DNA vaccines interfere in the regulation of established EAE. Lewis rats (6 per group) were immunized with MBPp68-86/CFA to induce active EAE, five, six and seven days later injected with either IP-10 or soluble β-actin encoding DNA vaccines (300 μg/rat per injection) and monitored for the development and progression of disease by an observer blind to the experimental procedure. The results are shown as mean maximal score±SE. Sixteen days after disease induction blood sera were obtained from 3 representative rats per group and determined for anti-IP-10 and anti-β actin specific antibody titers. Results are shown as mean log₂ antibody titer of 3 samples±SE.

FIG. 7 demonstrates that demonstrate that IP-10 encoding DNA vaccines can be used to treat established EAE. C57/BL mice were subjected to active induction of EAE. On day 15 these mice were separated into four groups of equally sick mice (six mice per group) and subjected to a repeated administration (3 times, days 15, 16, 17) of either IP-10 or soluble β-actin encoding DNA vaccines, empty vector (100 μg each) or PBS. The results are shown as mean maximal score±SE.

### DESCRIPTION OF THE PREFERRED EMBODIMENTS

The present invention is of methods and pharmaceutical compositions which can be used for breaking-down immunological tolerance the CXC chemokine interferon gamma-inducible protein 10 (IP-10), resulting in the generation of self specific immunity to IP-10, for the treatment of diseases, such as autoimmune diseases, in which IP-10 plays a pivotal role in disease onset and/or progression. Specifically, the present invention can be used to induce protective immunity against multiple sclerosis, so as to prevent or treat multiple sclerosis.

The principles and operation of the methods and compositions according to the present invention may be better understood with reference to the drawings and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

While reducing the present invention to practice, as is described in detail in the Examples section that follows, a significant anti-self response to IP-10 in rats immunized with MBPp68-86/CFA to induce active EAE, but not with CFA alone to induce a local inflammatory response was observed. This response accelerates in EAE rats following DNA vaccination to provide protective immunity. This type of intervention was turned on rapidly and could effectively suppress an established disease.

The mechanistic basis of the adjuvant effect of naked DNA vaccines with CpG motif has been solved very recently (88, 89). Either by activating Toll-like receptor 9 (TLR9) (88), and/or via the catalytic subunit of DNA-PKcs (89), a CpG motif initiates a signal transduction cascade in antigen presenting cells (e.g., dendritic cells) resulting in their activation. An abundant use of naked DNA vaccines with plasmids barring this motif is for the increase of the pro-inflammatory (Th1) immune response against infectious agents such as tuberculosis, HIV and allergens such as mite proteins (15, 39, 106-110). Interestingly, repeated administrations of DNA vaccines that include the CpG motif did not ameliorate T cell mediated autoimmunity in some previous experiments (90, 92-96, 17, 111). A recent observations suggest that CpG selects in vivo a low TNF-α producing subtype of CD4+ “Th1” cells (S. Youssef, G. Wildbaum and N. Karin, In preparation). This can explain, in part, why “pro-Th1” DNA vaccines do not aggravate autoimmunity. Hence, it is demonstrated herein, for the first time, how a CpG containing DNA vaccine could be constructed to preferentially select high IL-4, low IFN-γ, low TNF-α producing T cells. Such a construct could be used alone or together with a construct encoding a self autoimmune target antigen (such as MBP, PLP or MOG for MS) to effectively direct regulatory T cells to an autoimmune site.

Recent studies demonstrated the ability of IP-10 to stimulates the directional migration of activated T cells, particularly Th1 cells (97-99). Neutralizing the in vivo activity of IP-10 could affect T cell balance either because less Th1 cell are accumulated at the autoimmune site and/or because IP-10 is directly involved in T cell polarization. It is still an opened question, however, whether IP-10 may shift the Th1 Th2 balance towards Th2 as a result of a direct effect on T cell polarization. The data presented herein, showing that anti-IP-10 specific antibodies alter the in vivo balance towards high IL-4, low IFN-γ producing T cells (44% vs 18%) indicates that IP-10 is a chemokine with a dual function. Interestingly, it was previously shown that neutralizing antibodies to IL-18 not only can direct the in vivo polarization of autoimmune T cell and thus suppress EAE, but also the in vivo polarization of primary T cells (105).

Thus, according to one aspect of the present invention there is provided a method of breaking-down an immunological tolerance to interferon gamma-inducible protein 10 in a subject. The method is effected by administering to, or expressing within, the subject an amount of interferon gamma-inducible protein 10, or an immunological portion thereof, sufficient to elicit sufficient anti-interferon gamma-inducible protein 10 antibodies so as to break-down the immunological tolerance to interferon gamma-inducible protein 10. Alternatively, the method is effected by direct administration of anti-interferon gamma-inducible protein 10 antibodies so as to break-down the immunological tolerance to interferon gamma-inducible protein 10.

As used herein, the phrase “breaking-down an immunological tolerance” refers to generating self specific immunity against a self component.

As used herein, the term “subject” refers to an animal having an immune system, preferably a mammal, such as a human being, household pets, farm animals and mammals held in captivity.

Hence, according to another aspect of the present invention there is provided a method of generating self specific immunity to interferon gamma-inducible protein 10 in a subject. The method is effected by administering to, or expressing within, the subject an amount of interferon gamma-inducible protein 10, or an immunological portion thereof, sufficient to elicit sufficient anti-interferon gamma-inducible protein 10 antibodies so as to generate self specific immunity to interferon gamma-inducible protein 10. Alternatively, the method is effected by directly administering anti-interferon gamma-inducible protein 10 antibodies so as to generate self specific immunity to interferon gamma-inducible protein 10.

As used herein, the phrase “interferon gamma-inducible protein 10, or an immunological portion thereof” refers to the entire interferon gamma-inducible protein 10 protein and also to a peptide portion thereof which includes at least one continuous or discontinuous immunogenic epitope.

As used herein the term “peptide” includes native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body, or more immunogenic. Such modifications include, but are not limited to, cyclization, N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modification and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further detail in this respect are provided hereinunder.

Thus, a peptide according to the present invention can be a cyclic peptide. Cyclization can be obtained, for example, through amide bond formation, e.g., by incorporating Glu, Asp, Lys, Orn, di-amino butyric (Dab) acid, di-aminopropionic (Dap) acid at various positions in the chain (—CO—NH or —NH—CO bonds). Backbone to backbone cyclization can also be obtained through incorporation of modified amino acids of the formulas H—N((CH₂)ₙ—COOH)—C(R)H—COOH or H—N((CH₂)ₙ—COOH)—C(R)H—NH₂, wherein n=1-4, and further wherein R is any natural or non-natural side chain of an amino acid.

Cyclization via formation of S—S bonds through incorporation of two Cys residues is also possible. Additional side-chain to side chain cyclization can be obtained via formation of an interaction bond of the formula —(—CH₂—)ₙ—S—CH₂—C—, wherein n 1 or 2, which is possible, for example, through incorporation of Cys or homoCys and reaction of its free SH group with, e.g., bromoacetylated Lys, Orn, Dab or Dap.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), O-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

Tables 1-2 below list all the naturally occurring amino acids (Table 1) and non-conventional or modified amino acids (Table 2).

TABLE 1

| | Three-Letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| Isoleucine | Iie | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any amino acid as above | Xaa | X |

TABLE 2

| Non-conventional amino acid | Code | Non-conventional amino acid | Code |
|---|---|---|---|
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalaine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl | Nmbc | | |
| ethylamino)cyclopropane | | | |

A peptide according to the present invention can be used in a self standing form or be a part of moieties such as proteins and display moieties such as display bacteria and phages.

Additionally, a peptide according to the present invention includes at least five, optionally at least six, optionally at least seven, optionally at least eight, optionally at least nine, optionally at least ten, optionally at least eleven, optionally at least twelve, optionally at least thirteen, optionally at least fourteen, optionally at least fifteen, optionally at least sixteen or optionally at least seventeen, optionally between seventeen and twenty five or optionally between twenty five and at least thirty amino acid residues (also referred to herein interchangeably as amino acids).

Accordingly, as used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and omithine. Furthermore, the term “amino acid” includes both D- and L-amino acids.

The peptides of the invention can be derived from a specified protein or proteins and further from homologous regions of proteins homologous to the specified proteins of the same or other species, provided that these peptides are therapeutically effective. The term further relates to permissible amino acid alterations and peptidomimetics designed based on the amino acid sequence of the specified proteins or their homologous proteins.

As used herein the term “epitope” refer to a region of a molecule, such as, for example, the peptide(s) of the present invention, which region is characterized by specific molecular arrangement so as to elicit an immunological response thereto. When derived from a molecule which is linear by nature, yet acquires a complex three dimensional structure in which regions which are distant from one another in the linear topography are close to one another in the complex three dimensional structure, such as a protein, an epitope can either be continuous, i.e., defined by a contiguous sequence, or discontinuous, i.e., defined by a combination of at lest two non-contiguous regions of the sequence.

According to an additional aspect of the present invention there is provided a method of preventing or treating an autoimmune disease in which activity of interferon gamma-inducible protein 10 is pivotal in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to treat or prevent the autoimmune disease. Alternatively, the method comprising anti-interferon gamma-inducible protein 10 antibodies so as to treat or prevent the autoimmune disease.

As used herein, the term “treat” includes substantially inhibiting, slowing or reversing the progression of a disease, substantially ameliorating clinical symptoms of a disease or substantially preventing the appearance of clinical symptoms of a disease.

There are several autoimmune diseases in which activity of interferon gamma-inducible protein 10 is pivotal. These include, for example, multiple sclerosis, the pivotality of interferon gamma-inducible protein 10 in its development and progression is exemplified herein. Worth mentioning in this respect is that IP-10 was found to be highly expressed in demyelinating brain lesions of multiple sclerosis patients (113), the insulin producing beta islands of diabetic NOD mice (102), liver biopsies of patients suffering from chronic hepatitis (114), and its chemokine receptors CXCR3 is highly expressed in Immunostaining of T cells in rheumatoid arthritis synovial fluid (115).

Thus, according to still an additional aspect of the present invention there is provided a method of restricting a polarization of myelin basic protein specific T cells into Th2 cells in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to restrict a polarization of myelin basic protein specific T cells into Th2 cells. Alternatively, the method comprising directly administering anti-interferon gamma-inducible protein 10 antibodies so as to restrict a polarization of myelin basic protein specific T cells into Th2 cells.

According to a specific embodiment, the present invention provides a method of inducing protective immunity against multiple sclerosis in a subject. This method is effected by administering to, or expressing within, the subject an amount of interferon gamma-inducible protein 10, or an immunological portion thereof, sufficient to elicit sufficient anti-interferon gamma-inducible protein 10 antibodies, so as to induce protective immunity against multiple sclerosis in the subject. Alternatively, the method is effected by directly administering anti-interferon gamma-inducible protein 10 antibodies, so as to induce protective immunity against multiple sclerosis in the subject.

According to still another aspect of the present invention there is provided a method of preventing or treating multiple sclerosis in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to treat or prevent multiple sclerosis. Alternatively, the method comprising directly administering anti-interferon gamma-inducible protein 10 antibodies so as to treat or prevent multiple sclerosis.

In one of its embodiments, the present invention is practiced by administration of anti IP-antibodies to a subject in need. In the Examples section that follows anti-IP-10 antibodies elicited via DNA vaccination were affinity purified and administered to EAE rats, resulting in prevention of disease development and/or progression.

As used herein in the specification and in the claims section below, the term “antibody” refers to any monoclonal or polyclonal immunoglobulin, or a fragment of an immunoglobin such as sFv (single chain antigen binding protein), Fab1 or Fab2. The immunoglobulin could also be a “humanized” antibody, in which antibody variable regions of an animal (e.g., murine) are fused to human constant regions, or in which complementarity-determining regions are grafted onto a human antibody structure (Wilder, R. B. et al., J. Clin. Oncol., 14:1383-1400, 1996). Unlike mouse or rabbit antibodies, “humanized” antibodies often do not undergo an undesirable reaction with the immune system of the subject. The terms “sFv” and “single chain antigen binding protein” refer to a type of a fragment of an immunoglobulin, an example of which is sFv CC49 (Larson, S. M. et al., Cancer, 80:2458-68, 1997).

The elicitation and subsequent production of antibodies according to the present invention is through in vivo or in vitro techniques, the antibody having been prepared by a process comprising the steps of (a) exposing cells capable of producing antibodies to the IP-10 protein or the immonological portion thereof and thereby generating antibody producing cells; (b) immortalizing the antibody producing cells by viral transformation of by fusing the antibody producing cells with myeloma cells and thereby generating a plurality of immortalized cells each producing monoclonal antibodies; and (c) screening the plurality of monoclonal antibodies to identify a monoclonal antibody which specifically binds IP-10.

A clone that produces high amounts of efficient anti-IP-10 monoclonal antibody is than propagated and used to produce large amounts of the antibody. The antibody is preferably affinity purified against the IP-10 protein. The genes encoding the antibody can be clones and manipulated using techniques well known in the art so as to generate the single chain and/or humanized antibodies.

Gene therapy as used herein refers to the transfer of genetic material (e.g., DNA or RNA) of interest into a host to treat or prevent an acquired disease or condition or phenotype. The genetic material of interest encodes a protein product whose production in vivo is desired. For review see, in general, the text “Gene Therapy” (Advanced in Pharmacology 40, Academic Press, 1997).

Two basic approaches to gene therapy have evolved: (i) ex vivo and (ii) in vivo gene therapy. In ex vivo gene therapy cells are removed from a patient, and while being cultured are treated in vivo. Generally, a functional sequence is introduced into the cell via an appropriate gene delivery vehicle/method (transfection, transduction, homologous recombination, etc.) and an expression system as needed and then the modified cells are expanded in culture and returned to the host/patient. These genetically reimplanted cells have been shown to express the transfected genetic material in situ.

In in vivo gene therapy, target cells are not removed from the subject rather the genetic material to be transferred is introduced into the cells of the recipient organism in situ, that is within the recipient. These genetically altered cells have been shown to express the transfected genetic material in situ.

The gene expression construct is capable of delivery/transfer of heterologous nucleic acid into a host cell. The expression construct may include elements to control targeting, expression and transcription of the nucleic acid in a cell selective manner as is known in the art. It should be noted that often the 5′UTR and/or 3′UTR of the gene may be replaced by the 5′UTR and/or 3′UTR of the expression construct. Therefore, as used herein the expression construct may, as needed, not include the 5′UTR and/or 3′UTR of the actual gene to be transferred and only include the specific amino acid coding region.

The expression construct can include a promoter for controlling transcription of the heterologous material and can be either a constitutive or inducible promoter to allow selective transcription. Enhancers that may be required to obtain necessary transcription levels can optionally be included. Enhancers are generally any nontranslated DNA sequence which works contiguously with the coding sequence (in cis) to change the basal transcription level dictated by the promoter. The expression construct can also include a selection gene as described herein below.

Expression constructs can be introduced into cells or tissues by any one of a variety of known methods within the art. Such methods can be found generally described in Sambrook et al., Molecular Cloning: A Laboratory Manual, Cold Springs Harbor Laboratory, New York 1989, 1992), in Ausubel et al., Current Protocols in Molecular Biology, John Wiley and Sons, Baltimore, Md. 1989), Chang et al., Somatic Gene Therapy, CRC Press, Ann Arbor, Mich. 1995), Vega et al., Gene Targeting, CRC Press, Ann Arbor Mich. (995), Vectors: A Survey of Molecular Cloning Vectors and Their Uses, Butterworths, Boston Mass. 1988) and Gilboa et al. (Biotechniques 4 (6): 504-512, 1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

In some cases, introduction of nucleic acids by infection offers several advantages over the other listed methods. Higher efficiency can be obtained due to their infectious nature. Moreover, viruses are very specialized and typically infect and propagate in specific cell types. Thus, their natural specificity can be used to target the vectors to specific cell types in vivo or within a tissue or mixed culture of cells. Viral vectors can also be modified with specific receptors or ligands to alter target specificity through receptor mediated events.

A specific example of DNA viral vector introducing and expressing recumbent sequences is the adenovirus-derived vector Adenop53TK. This vector expresses a herpes virus thymidine kinase (TK) gene for either positive or negative selection and an expression cassette for desired recombinant sequences. This vector can be used to infect cells that have an adenovirus receptor. This vector as well as others that exhibit similar desired functions can be used to treat a mixed population of cells and can include, for example, an in vivo or ex vivo culture of cells, a tissue or a human subject.

Features that limit expression to particular cell types can also be included. Such features include, for example, promoter and regulatory elements that are specific for the desired cell type.

In addition, recombinant viral vectors are useful for in vivo expression of a desired nucleic acid because they offer advantages such as lateral infection and targeting specificity. Lateral infection is inherent in the life cycle of, for example, retrovirus and is the process by which a single infected cell produces many progeny virions that bud off and infect neighboring cells. The result is that a large area becomes rapidly infected, most of which was not initially infected by the original viral particles. This is in contrast to vertical-type of infection in which the infectious agent spreads only through daughter progeny. Viral vectors can also be produced that are unable to spread laterally. This characteristic can be useful if the desired purpose is to introduce a specified gene into only a localized number of targeted cells.

Retroviral vectors can be constructed to function either as infectious particles or to undergo only a single initial round of infection. In the former case, the genome of the virus is modified so that it maintains all the necessary genes, regulatory sequences and packaging signals to synthesize new viral proteins and RNA. Once these molecules are synthesized, the host cell packages the RNA into new viral particles which are capable of undergoing further rounds of infection. The vector's genome is also engineered to encode and express the desired recombinant gene. In the case of non-infectious viral vectors, the vector genome is usually mutated to destroy the viral packaging signal that is required to encapsulate the RNA into viral particles. Without such a signal, any particles that are formed will not contain a genome and therefore cannot proceed through subsequent rounds of infection. The specific type of vector will depend upon the intended application. The actual vectors are also known and readily available within the art or can be constructed by one skilled in the art using well-known methodology.

The recombinant vector can be administered in several ways. If viral vectors are used, for example, the procedure can take advantage of their target specificity and consequently, do not have to be administered locally at the diseased site. However, local administration can provide a quicker and more effective treatment, administration can also be performed by, for example, intravenous or subcutaneous injection into the subject. Injection of the viral vectors into a spinal fluid can also be used as a mode of administration. Following injection, the viral vectors will circulate until they recognize host cells with appropriate target specificity for infection.

Although, the most common problems encountered in prior art gene therapy protocols are poor efficacy and immune response of the host to the vector, these problems are of lesser or no influence while practicing the present invention as following a brief period of expression immunological memory develops.

Thus, according to the present invention, expressing within the subject the amount of interferon gamma-inducible protein 10 can be effected by generating in (in vivo gene therapy), or introducing into (ex vivo gene therapy), the subject cells expressing recombinant interferon gamma-inducible protein 10, or an immunological portion thereof.

Such cells can be removed from the subject, transformed with an expression construct having a strong promoter for directing gene expression and which encodes the interferon gamma-inducible protein 10, or the immunological portion thereof and once a sufficient level of expression is detected, re-introduced into the subject from which they were derived. Therein, such cells will express and secrete the interferon gamma-inducible protein 10, or the immunological portion thereof.

In the alternative, the expression construct can be used to directly vaccinate the subject, as is further exemplified and described herein.

The expression construct used while implementing the invention can be a viral eukaryotic expression vector ad described above of a naked DNA construct suitable for DNA vaccination, such as, but not limited to, pcDNA3, pcDNA3.1(+/−), pZeoSV2(+/−), pSecTag2, pDisplay, pEF/myc/cyto, pCMV/myc/cyto, pCR3.1, which are available from Invitrogen, pCI which is available from Promega, pBK-RSV and pBK-CMV which are available from Stratagene, pTRES which is available from Clontech, and their derivatives.

Thus, according to a presently preferred embodiment of the present invention generating in the subject cells expressing recombinant interferon gamma-inducible protein 10, or an immunological portion thereof, is effected by vaccinating the subject with an expression construct encoding interferon gamma-inducible protein 10, or the immunological portion thereof.

As already mentioned herein above, the expression construct includes transcription control sequences of any suitable type compatible with eukaryotic gene expression. Strong and effective control sequences are preferably of choice. These sequences can be from a mammalian or viral source. Examples include, but are not limited to, RSV control sequences, CMV control sequences, retroviral LTR sequences, SV-40 control sequences and β-actin control sequences, myosin control sequences, all of which are potent and effective control sequences, capable of efficiently directing gene expression in either a plurality of cell types of specific cell types (e.g., tissue specific promoters).

The in vivo level of expression of the interferon gamma-inducible protein 10, or the immunological portion thereof can be readily monitored using serum samples derived from the treated subject. Such serum samples can be analyzed for the level of interferon gamma-inducible protein 10, or the immunological portion thereof using assays well known to the skilled artisan, including, but not limited to, enzyme linked immunosorbent assay (ELISA), immunopercipitation, Western blots, slot and dot blots, magnetic bead separation, solid support arrays, affinity columns and phage or bacterial display assays. These methods are well known in the art and as such no further description thereof is provided herein.

The amount of interferon gamma-inducible protein 10, or an immunological portion thereof, sufficient to elicit sufficient anti-interferon gamma-inducible protein 10 antibodies can be determined experimentally for various diseases, using experimental procedures similar to those described in detail in the Examples section that follows and as is further described hereinunder with respect to the pharmaceutical compositions of the present invention.

The amount of circulating anti-interferon gamma-inducible protein 10 antibodies required to prevent or treat a disease is determinable using similar experimental procedures.

A pharmaceutical formulation according to the present invention includes, as an active ingredient, an amount of interferon gamma-inducible protein 10, or an immunological portion thereof, or of an expression construct encoding said interferon gamma-inducible protein 10, or said immunological portion thereof, sufficient to elicit sufficient anti-interferon gamma-inducible protein 10 antibodies, or the anti-interferon gamma-inducible protein 10 antibodies themselves in an amount sufficient to achieve a desired therapeutic effect in the subject. The active ingredients can be administered to an organism per se, or in a pharmaceutical composition where it is mixed with suitable carriers or excipients.

Thus, each of the pharmaceutical compositions of the invention comprises, a pharmaceutically acceptable carrier approved for medical or veterinary administration and, as an active ingredient, an amount of interferon gamma-inducible protein 10, an immunological portion thereof, or of an expression construct encoding said interferon gamma-inducible protein 10, or said immunological portion thereof sufficient to elicit sufficient anti-interferon gamma-inducible protein 10 antibodies, or the anti-interferon gamma-inducible protein 10 antibodies themselves in an amount sufficient to achieve a desired therapeutic effect in the subject.

The pharmaceutical compositions of the invention can be used for (i) breaking-down an immunological tolerance to interferon gamma-inducible protein 10 in a subject; (ii) generating self specific immunity to interferon gamma-inducible protein 10 in a subject; (iii) preventing or treating an autoimmune disease in which activity of interferon gamma-inducible protein 10 is pivotal, in a subject; (iv) restricting a polarization of myelin basic protein specific T cells into Th2 cells in a subject; (v) preventing or treating multiple sclerosis in a subject; and for (vi) inducing protective immunity against multiple sclerosis in a subject.

As used herein a “pharmaceutical composition” refers to the active ingredients as described herein mixed with other chemical components such as physiologically suitable carriers and excipients. The purpose of a pharmaceutical composition is to facilitate administration of a compound to an organism.

Hereinafter, the terms “physiologically acceptable carrier” and “pharmaceutically acceptable carrier” which may be interchangeably used refer to a carrier or a diluent that does not cause significant irritation to an organism and does not abrogate the biological activity and properties of the administered compound.

Herein the term “excipient” refers to an inert substance added to a pharmaceutical composition to further facilitate administration of a compound. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, intestinal or parenteral delivery, including intramuscular, subcutaneous and intramedullary injections as well as intrathecal, direct intraventricular, intravenous, inrtaperitoneal, intranasal, or intraocular injections.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active compounds into preparations which, can be used pharmaceutically. Proper formulation is dependent upon the route of administration chosen.

For injection, the compounds of the invention may be formulated in aqueous solutions, preferably in physiologically compatible buffers such as Hank's solution, Ringer's solution, or physiological saline buffer. For transmucosal administration, penetrants appropriate to the barrier to be permeated are used in the formulation. Such penetrants are generally known in the art.

For oral administration, the active ingredients can be formulated readily by combining with pharmaceutically acceptable carriers well known in the art. Such carriers enable the active ingredients to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The preparations described herein may be formulated for parenteral administration, e.g., by bolus injection or continues infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water, before use.

The preparation of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase carriers or excipients. Examples of such carriers or excipients include, but are not limited to, calcium carbonate, calcium phosphate, various sugars, starches, cellulose derivatives, gelatin and polymers such as polyethylene glycols.

Pharmaceutical compositions of the invention preferably include an immunization adjuvant approved by a regulatory entity such as the FDA for medical and/or veterinary use.

Pharmaceutical compositions suitable for use in context of the present invention include compositions wherein the active ingredients are contained in an amount effective to achieve the intended purpose. More specifically, a therapeutically effective amount means an amount of the active ingredient effective to prevent, alleviate or ameliorate symptoms of disease or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from animal models. Such information can be used to more accurately determine useful doses in humans.

Toxicity and therapeutic efficacy of the active ingredients described herein can be determined by standard pharmaceutical procedures in experimental animals, e.g., by determining the LD₅₀ (lethal dose causing death in 50% of the tested animals). The data obtained from these animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma levels of the active antibodies which are sufficient to create immunological memory.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition. Suitable conditions indicated on the label may include treatment of diseases in which interferon gamma-inducible protein 10 plays a pivotal role, such as autoimmune disease, including multiple sclerosis, rheumatoid arthritis and other autoimmune diseases accompanied by inflammation in which IP-10 has a pivot function.

In general, the present invention is practiced by directly (i.e., administration of anti-IP-10 antibodies) or indirectly (administration of IP-10 or a construct capable of in vivo expression of IP-10) introducing anti-interferon gamma-inducible protein 10 antibodies to the subject in an amount sufficient to treat or prevent a disease, syndrome or a manifestation associated therewith.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N.Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

Materials and Experimental Procedures

Rats:

Female Lewis rats, approximately six weeks old were purchased from Harlan (Jerusalem, Israel) and maintained under SPF conditions in an animal facility.

Peptide Antigens:

Myelin Basic Protein (MBP) p68-86, Y G S L P Q K S Q R S Q D E N P V (SEQ ID NO:1), was synthesized on a MilliGen 9050 peptide synthesizer by standard 9-fluorenylmethoxycarbonyl chemistry and purified by high performance liquid chromatography. Structure was confirmed by amino acid analysis and mass spectroscopy. Only peptides that were greater than 95% pure were used in subsequent experiments.

Immunizations and Active Disease Induction:

Rats were immunized subcutaneously in the hind foot pads with 0.1 ml of MBP epitope 68-86 (p68-86) dissolved in PBS (1 mg/ml) and emulsified with an equal volume of CFA (incomplete Freund's adjuvant supplemented with 4 mg/ml heat-killed Mycobacterium tuberculosisH37Ra in oil (Difco laboratories, Inc., Detroit, Mich.). Rats were then monitored for clinical signs daily by an observer blind to the treatment protocol. EAE was scored as follows: 0, clinically normal; 1, flaccid tail; 2, hind limb paralysis; 3, total hind limb paralysis, accompanied by an apparent front limb paralysis; and 4, total hind limb and front limb paralysis.

Reverse Transcriptase Polymerase Chain Reaction (RT-PCR) Analysis: RT-PCR analysis was utilized on brain samples according to a protocol described elsewhere (94). IP-10 specific oligonucleotide primers were designed based on its published sequence (NCBI Accession Number: U22520) as follows: Rat IP-10 sense 5′-CATGAACCCAAGTGCTGCTGTCGT-3′ (SEQ ID NO:2); and Rat IP-10 antisense 5′-TTACGGAGCTCTTTTAGACCTTCT-3′ (SEQ ID NO:3). The PCR product was then cloned and its sequence was verified as described below.

Cloning and Sequencing of PCR Products:

The PCR product described above was cloned into a pUC57/T vector (T-cloning Kit, Cat. No. K1212, MBI Fermentas, Lithuania) and transformed into E. Coli according to the manufacturer's protocol. Each clone was then sequenced (Sequenase Version 2, USB, Cleveland, Ohio) according to the manufacturer's protocol.

DNA Vaccination:

DNA vaccination was performed as previously described (94). A sequenced PCR product of rat IP-10 was transferred into a pcDNA3 vector (Invitrogen, San Diego, Calif.). Large scale preparation of plasmid DNA was conducted using Mega prep (Qiagen Inc., Chatsworth, Calif.). Cardiotoxin (Sigma, St. Louis, Mo.) was injected into the tibialis anterior muscle of 4-6 weeks old female Lewis rats (10 μM per leg). One week following injection rats were injected with 100 μg DNA in PBS. Four-five days after the first immunization one rat from the group previously subjected to IP-10 DNA vaccination was sacrificed and transcription of IP-10 was verified using RT-PCR on tibialis anterior muscle samples. Thereafter, identical doses of naked DNA vaccines were given 3-5 times with intervals of 6-7 days between each injection.

Production and Purification of Recombinant IP-10:

PCR product was recloned into a PQE expression vector, expressed in E. Coli (Qaigen, Chatsworth, Calif.) and then purified by an NI-NTA-supper flow affinity purification of 6xHis proteins (Qaigen, Chatsworth, Calif.). After purification the purity of the recombinant IP-10 was verified by gel electrophoresis. The recombinant protein sequence was verified (N-terminus).

Western Blot Analysis:

The recombinant rat IP-10, produced as described above, and commercially available recombinant mouse IP-10 (Cytolab, Rehovot, Israel) were each subjected to Western blot analysis according to the protocol described in details elsewhere (112) with a minor modification of using a 12% (rather than 8%) running gel. IgG from IP-10 DNA vaccinated rats, or IgG from normal rat serum (final concentration of 1:500 each) were used as primary antibodies. A goat anti-rat alkaline phosphates conjugated antibody (Sigma) was used a secondary antibody. BCIP (Sigma, 0.15 mg/ml) and NBT (Sigma, 0.3 mg/ml) were then used as a substrate.

Evaluation of Anti-IP-10 Ligand Antibody Titer in Sera of DNA Vaccinated Rats:

A direct ELISA assay has been utilized to determine the anti-IP-10 antibody titer in DNA vaccinated rats. The recombinant IP-10 was coated onto 96 well ELISA plates (Nunc, Denmark), at a concentration of 50 ng/well. Rat anti-sera, in serial dilutions of from 2⁸ to 2³⁰ were added to the wells of the ELISA plates. A goat anti-rat IgG alkaline phosphatase conjugated antibody (Sigma) was used as a secondary antibody p-Nitrophenyl Phosphate (p-NPP) (Sigma) was used as a soluble alkaline phosphatase substrate. The results are shown as log 2 antibody titer±SE.

CNBr Purification of Anti-IP-10 Specific Antibodies:

Recombinant rat IP-10 (5 mg) was bound to a CNBr activated Sepharose column according to the manufactures instructions (Pharmacia biotech, Cat. No. 17-0820-01). Anti-IP-10 specific antibodies from sera (IgG fraction) of DNA vaccinated rats were loaded on the column and then eluted by an acidic elution buffer (glycine, pH 2.5). Isotype determination of the purified antibody (ELISA) revealed that purified antibodies are mostly of the IgG2a Isotype.

Cytokine Determination in Cultured Primary Spleen Cells:

Spleen cells from EAE donor rats were stimulated in vivo (10⁷ cells/ml) in 24 well plates (Nunc) with 100 μM p68-86. After 72 hours of stimulation, supernatants were assayed for the protein level of various cytokines using semi-ELISA kits:

1. IFN-γ: rabbit anti-rat IFN-γ polyclonal antibody (CY-048, Innogenetics, Belgium) as a capture antibody, biotinylated mouse anti-rat monoclonal antibody (CY-106 clone BD-1, Innogenetics) as a detection antibody, and alkaline phosphatase-streptavidin (Cat No. 43-4322, Zymed, SF, CA) with rat recombinant IFN-γ as a standard (Cat. No. 3281 SA, Life Technologies).

2. TNF-α: commercial semi-ELISA kit for the detection of rat TNF-α, (Cat. No. 80-3807-00, Genzyme, Cambridge, Mass.).

3. IL-4: mouse anti-rat IL-4 monoclonal antibody (24050D OX-81, PharMingen, San Diego, Calif.) as a capture antibody, and rabbit anti-rat IL-4 biotin-conjugated polyclonal antibody (2411-2D, PharMingen) as a secondary antibody. Recombinant rat IL-4, purchased from R&D (504-RL), was used as a standard.

4. IL-10: commercial semi-ELISA kits for the detection of rat IL-10 (PharMingen, San Diego, Calif.).

FACS Analysis:

FACS analysis was conducted according to the basic protocol described in details elsewhere (91). Intracellular staining of IFN-γ and IL-4 was done using a commercially available kit (LEUCOPERM, Serotec, Oxford, UK, Cat. No. BUF9) according to the manufacturer's protocol. A FITC labeled mouse anti-rat IFN-γ monoclonal antibody (Biosource, Nivelles, Belgium) and PE labeled mouse anti-rat IL-4 monoclonal antibody (Biosource) were used for direct staining. Cells were analyzed using a FACSCalibur (Becton Dickinson, Mountain View, Calif.). Data were collected for 10,000 events and analyzed using a Cell Quest program (Becton Dickinson).

Histopathology:

Histological examinations of hematoxylin and eosin-stained, formalin-fixed, paraffin-embedded sections of the lower thoracic and lumbar regions of the spinal cord were performed. Each section was evaluated without prior knowledge of the treatment status of the animal.

The following scale was used: 0, no mononuclear cell infiltration; 1, 1 to 5 perivascular lesions per section with minimal parenchymal infiltration; 2, 5 to 10 perivascular lesions per section with parenchymal infiltration; and 3, over 10 perivascular lesions per section with extensive parenchymal infiltration. The mean histological score±SE was calculated for each treatment group.

Statistical analysis significance of differences was applied using the Student's t-test. A value of p<0.05 was considered significant. Mann-Whitney sum of ranks test was used to evaluate significance of differences in mean of maximal clinical score. Value of p<0.05 was considered significant.

### Experimental Results

IP-10 Encoding DNA Vaccine Redirect Antigen Specific T Cell Polarization and Suppresses EAE:

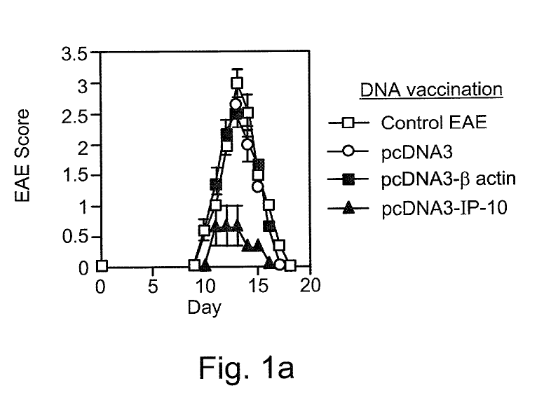

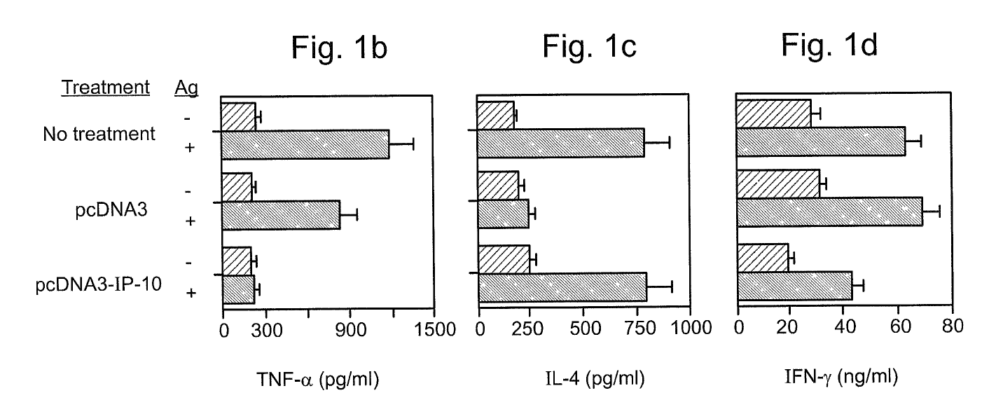

Cloned PCR products of rat IP-10 were ligated into a pcDNA3 eukaryotic expression vector and used as constructs for naked DNA vaccination. Rats were subjected to four weekly injections of the above construct. Control rats were injected either with the pcDNA3 vector alone, pcDNA3-β-actin encoding construct or with PBS. Two months after the last immunization all rats were immunized with p68-86/CFA to induce active EAE. All control groups developed active disease that persisted for 5-6 days (FIG. 1A, 6/6 in each group with a maximum clinical score 2.83±0.18, 2.5±0.23 and 2.33±0.23 in PBS, pcDNA3-β-actin and pcDNA3 immunized rats, respectively). In contrast, rats injected with the IP-10 naked DNA vaccine developed a markedly reduced degree of disease (FIG. 1 incidence of 6/6 with a maximum clinical score of 1.16±0.18, p<0.016 for the comparison of this treatment to PBS treated rats and p<0.02 for the comparison with each of the other control groups). Disease suppression was accompanied by a marked reduction in perivascular mononuclear cell infiltration, as was histologically observed in a double blind evaluation (Table 3, p<0.001).

TABLE 3
Targeted DNA vaccines encoding IP-10 markedly
decrease CNS mononuclear cell infiltration.

| | | Induction of | Mean Histological |
|---|---|---|---|
| | Treatment | EAE | Score |
| A | PBS | − | 0 ± 0 |
| B | PBS | + | 2.66 ± 0.3 |
| C | PcDNA3 alone | + | 2.33 ± 0.3 |
| D | PcDNA3-IP-10 | + | 0.66 ± 0.3* |
| *p < 0.001 for D compared with either B or C | | | |
| Table 3: | | | |
| At the peak of disease describrd above (FIG. 1A) 3 rats per group were secrifised and spinal cord samples were subjected to histological evaluation. The following scale was used: 0, no mononuclear cell infiltration; 1, 1 to 5 perivascular lesions per section with minimal parenchymal infiltration; 2. 5 to 10 perivascular lesions per section with parenchymal infiltration; and 3 >10 perivascular lesions per section with extensive parenchymal infiltration. The mean histological score of 18 | | | |
| #different sections ± SE was calculated for each treatment group. | | | |

Thus, the autoimmune response subsequent to administration of the IP-10 encoding naked DNA vaccine leads to a marked reduction in mononuclear cell infiltration to the CNS and suppresses EAE (FIGS. 1A-D and Table 3).

To determine whether IP-10 encoding DNA vaccine altered antigen specific T cell polarization, spleen T cells derived from the rats were cultured with MBPp68-86 and levels of Th1 Vs Th2 cytokines were determined. Rats administered with IP-10 encoding DNA vaccine showed a remarkable elevation in the ex vivo production IL-4 (FIG. 1C, p<0.0001 compared to each control group) together with a marked reduction in TNF-α production (FIG. 1B, p<0.0001 compared to control EAE rats and p<0.05 compared to pcDNA3 treated EAE rats) and a significant reduction in IFN-γ production (FIG. 1D, p<0.01 to all control groups). Based on these findings was hypothesized that a DNA vaccine encoding IP-10 leads to in vivo neutralization of IP-10 and thus alters T cell polarization into high IL-4, low IFN-γ, low TNF-α producing T cells. To explore this hypothesis several consecutive experiments were conducted as described bellow.

IP-Encoding DNA Vaccine Induces Breakdown of Tolerance to its Gene Product and Generates Immunity to Native IP-10:

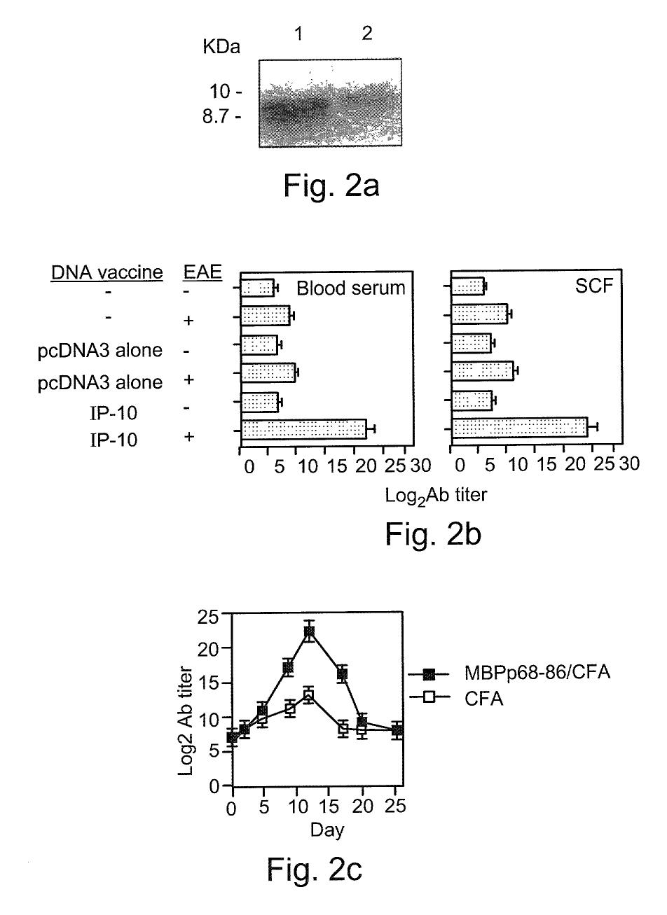

DNA vaccination can potentially elicit both cellular and humoral responses against products of a given construct. To determine whether the administration of IP-10 DNA vaccine leads to a breakdown of tolerance to self IP-10, Lewis rats were subjected to four weekly injections of the IP-10 DNA construct as described in FIGS. 2A-C. Two months after the last immunization, when IP-10 specific antibody titer retained a baseline level (log₂ antibody titer of 7-8 in both DNA vaccinated and control untreated rats), these rats, and rats treated with either the pcDNA3 alone or PBS, were injected with p68-86/CFA to induce active EAE. Representative rats from each group were injected with CFA alone. At different time points blood serum and spinal cord fluid (SCF) from representative rats were analyzed for the presence of antibodies to IP-10, first by Western blot analysis (FIG. 2A) and then by measuring log2 of antibody titer. SDS-PAGE under reducing conditions followed by Western blot analysis 1 revealed that both, these antibodies obtained from IP-10 DNA vaccinated rats bound a single 10 kDa fragment in supernatant of activated primary spleen T cells, and also a 8.7 kDa commercially available mouse IP-10 fragment (FIG. 2A). The sequence of the 10 kDa native rat IP-10 has been verified (N-terminus analysis), as described elsewhere (105). Thus, self specific antibodies generated in IP-10 DNA vaccinated rats bind the natural form of self IP-10. An elicited titer to the above gene product was observed both in blood serum and SCF of IP-10 DNA vaccinated rats eight days after disease induction. This titer reached its maximal level on day 13 (FIG. 2B, log₂ antibody titer of 22±1.2 and 24±2.6 in blood serum and SCF of IP-10 DNA vaccinated rats compared with 10±0.3 and 11±0.3 in blood serum and SCF of pcDNA3 vaccinated rats and with 7±0.2 in PBS treated EAE rats, p<0.001 for the compression of each control group with the respective IP-10 DNA vaccinated group). Taken together these results suggest that IP-10 naked DNA vaccination leads to a breakdown of immunological tolerance, resulting in the generation of an immunological memory which is turned on upon EAE induction (FIG. 2B). CFA immunization alone did significantly elicit the production of self specific antibodies to IP-10 in DNA vaccinated rats (FIG. 2C, in blood serum log₂ antibody titer of 13±0.5 compared with 7±0 without CFA immunization, p<0.01), though to a much lesser extent than an induction of active disease did (FIG. 2C). Similar results were also previously obtained when tolerance to self was broken using MIP-1α, MCP-1, TNF-α or FasL encoding DNA vaccines (90, 91, 93, 94, 96). The immunological basis of these differences is not fully understood yet.

IP-10-Specific Antibodies Generated in DNA Caccinated EAE rats are Neutralizing Antibodies That Inhibit Both Migratory Properties and Polarization of Activated T Cells:

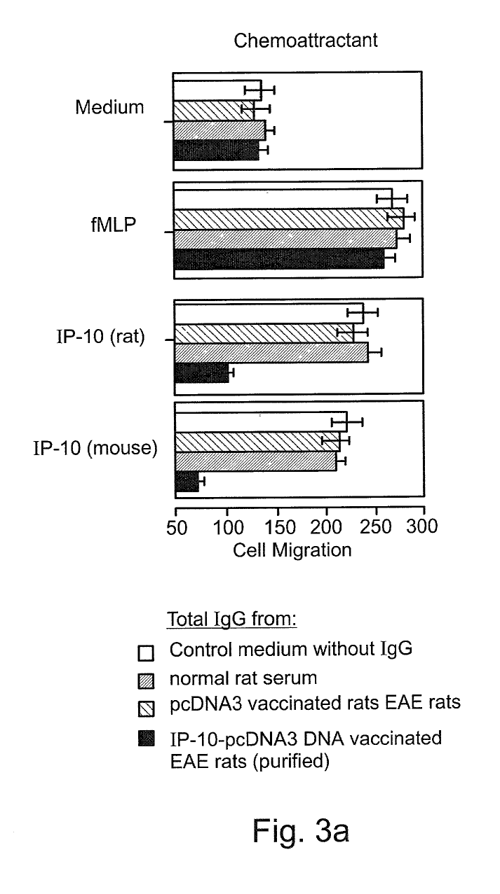

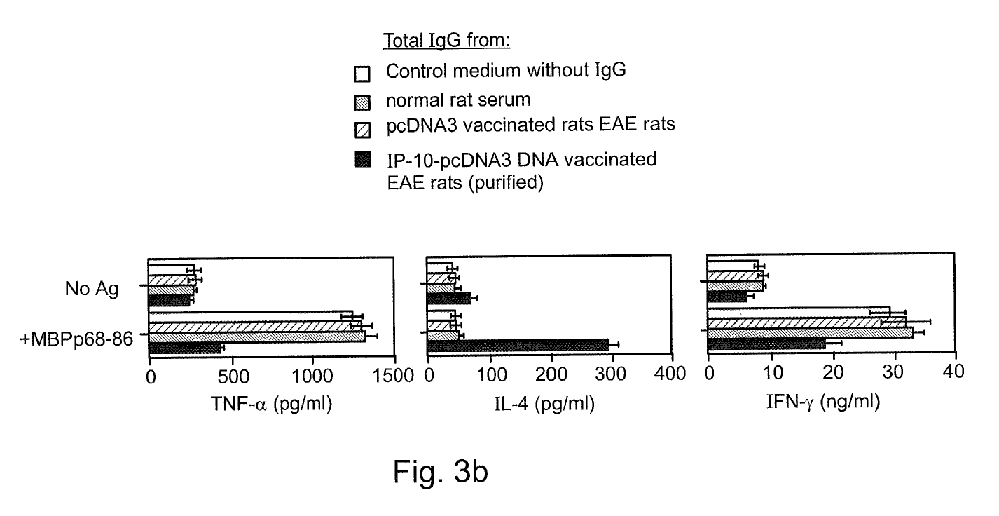

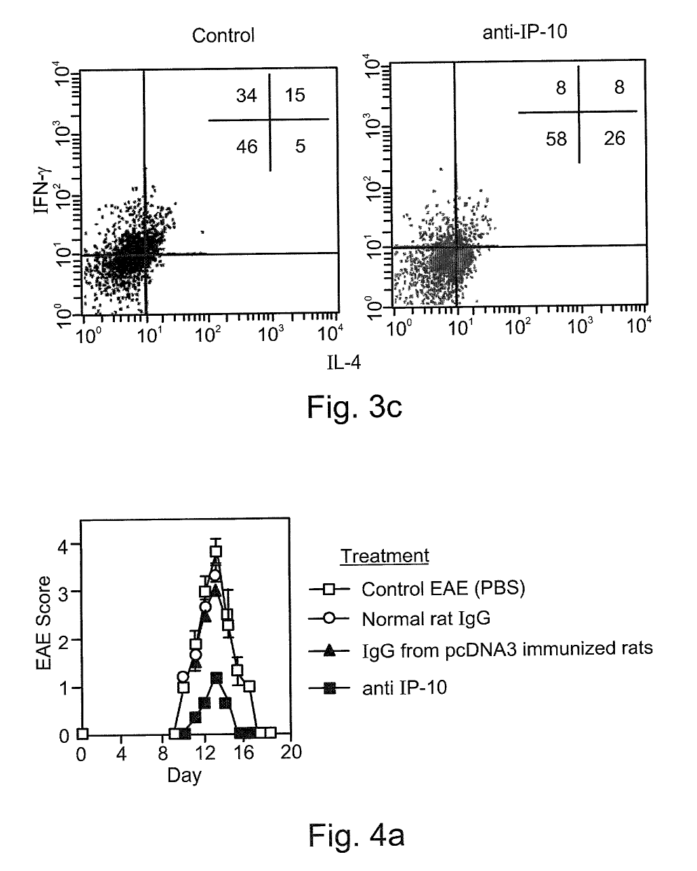

Since DNA vaccination can potentially elicit both cellular and humoral responses against products of a given construct, it is difficult to know which of these responses contributed more to the development of EAE resistance in IP-10 DNA vaccinated rats (FIGS. 1A-D). The possibility that self-specific antibodies generated in DNA vaccinated rats against IP-10 contribute to the tolerant state in IP-10 DNA vaccinated rats was thus explored. At first, the in vivo competence of these antibodies to inhibit the IP-10 induced migration of a MBP specific encephalitogenic CD4+ T cell line was determined in a Boyden chamber assay (FIG. 3A). Not only could these antibodies (CNBr purified) significantly inhibit encephalitogenic CD4+ T cell migration induced by recombinant rat IP-10, they could also prevent rat T cell migration induced by commercially (Cytolab, Rehovot, Israel) available 8.7 kDa fragment of the peptide (FIG. 3A, p<0.001). These antibodies had no effect on MCP-1 induced migration of these cells (data not shown). Thus the inhibitory effect of IP-10 self specific antibodies is chemokine specific. The addition of these antibodies to MBP specific (p68-86) cultured spleen cells led to a significant decrease in TNF-α and IFN-γ production (FIG. 3B, 1240±60 vs. 460±20 pg/ml, p<0.001, and 29.8±1.9 vs. 18.1±1.2 ng/ml, p<0.01 respectively). This reduction was accompanied by a marked increase in IL-4 production (FIG. 3B, 48±7 vs. 294±12 pg/ml, p<0.0001). Moreover, intracellular flow cytometry analysis (FIG. 3C) clearly showed an apparent shift from high IFN-γ, low IL-4 producing CD4+ T cells (34% high IFN-γ, low IL-4, 5% high IL-4, low IFN-γ) in cultures that were not supplemented with anti-IP-10 antibodies, to low IFN-γ, high IL-4 producing CD4+ T cells in the presence of these antibodies (8% high IFN-γ, low IL-4, 26% high IL-4, low IFN).

Taken together, the data shows that self specific antibodies to IP-10 affect both the migratory properties and the cytokine profile of autoreactive T cells. Differential counting (by FACS) of macrophages (ED1 positive), CD4+ and CD8+ cells ruled out the possibility that these antibodies are depleting antibodies (not shown).

Administration of Self Specific Antibodies to IP-10 Redirects Antigen Specific T Cell Polarization Towards Th2 and Suppresses EAE:

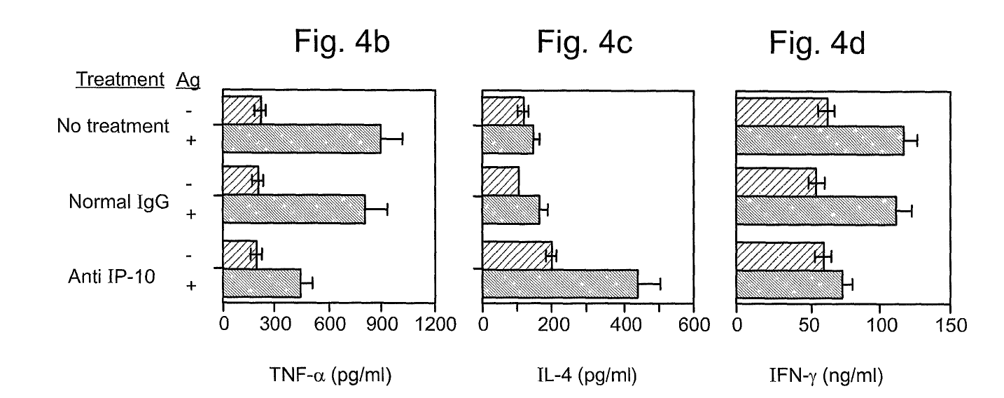

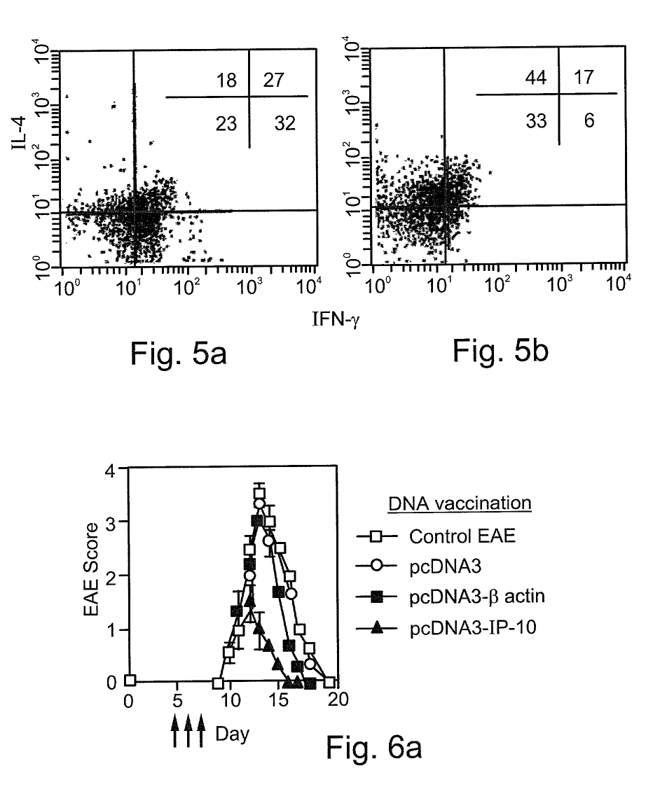

The in vivo properties of the self-specific anti-IP-10 antibodies may suggest that these antibodies may affect the in vivo function of autoimmune T cells and thus the regulation of EAE. The subsequent administration of these antibodies starting five days before the onset of disease led to a marked reduction in the clinical (FIG. 4A) and histological scores of disease. That is, while all control EAE rats immunized with p68-86/CFA and then treated with either PBS, or with IgG (protein G purified) from either naive or pcDN3 treated EAE rats, developed severe EAE (FIG. 4A, Mean Maximal score 3.83±0.25, 3.3±0.8 and 3±0.3) those treated with purified anti-IP-10 specific antibodies exhibited a mild form of the disease (mean maximal score of 1.16±0.23, p<0.001 compared to each control group). Disease inhibition was accompanied by a significant, but not total, reduction in parenchymal mononuclear cell infiltration (mean histological score of 0.83±0.16 vs 2.6±0.2 and 2.3±0.3 and 2.16±0.3 in control groups, respectively). To assess the possibility that the administration of anti-IP-10 specific antibodies affected antigen specific T cell polarization, primary spleen T cells from EAE rats treated with either PBS, normal rat IgG or anti-IP-10 antibodies were cultured with, or without, the MBP encephalitogenic determinant and supernatant levels of TNF-α (FIG. 4B), IL-4 (FIG. 4C) and IFN-γ (FIG. 4D) were determined. The addition of anti-IP-10 antibodies to MBP specific (p68-86) cultured spleen cells led to a significant decrease in TNF-α and IFN-γ production (p<0.001). This reduction was accompanied by a marked increase in IL-4 production (p<0.0001). To further asses the possibility that neutralizing IP-10 shifts the T cell subset balance towards Th2 CD4+, primary T cells form these cultured T cells were subjected to intra-cellular staining of IFN-γ and IL-4. About 60% of control CD4+T cells (from rats treated with normal IgG) included high IFN-γ producing Th1 (32%) and Th0 (27%) cells and only 18% of high IL-4 low IFN-γ producing “Th2” cells (FIG. 5A). In contrast, primary T cells (CD4+) from MBP activated spleen T cells from rats treated with anti-IP-10 antibodies exhibited a significant shift towards Th2 (FIG. 5B, 44% high IL-4, low IFN-γ producing T cells and only 6% high IFN-γ, low IL-4 producing cells).

IP-Encoding DNA Vaccines Interferes in the Regulation of Established EAE:

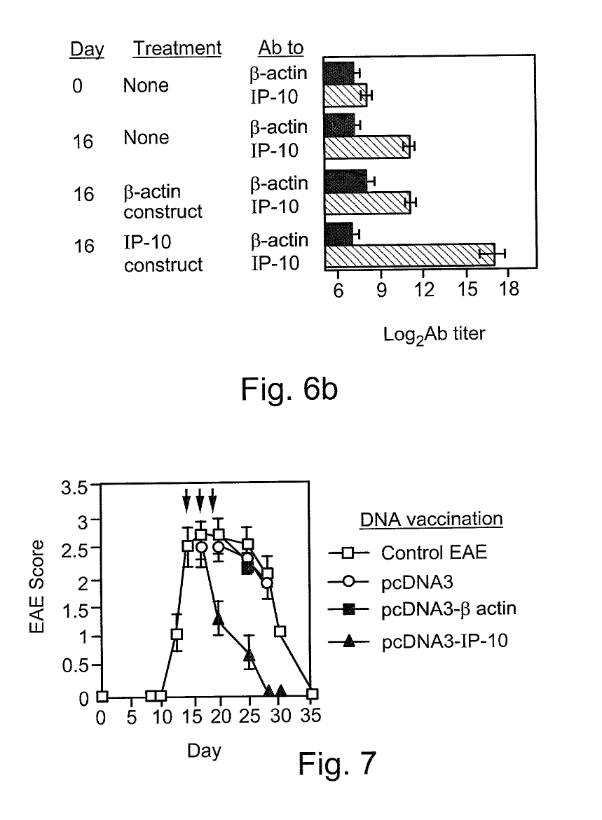

Finally, whether IP-10 encoding DNA vaccines may interfere in the regulation of established EAE was determined. Thus Lewis rats were immunized with MBPp68-86/CFA to induce active EAE, five, six and seven days later injected with either IP-10 or soluble β-actin encoding DNA vaccines (300 μg/rat per injection) and monitored for the development and progression of disease by an observer blind to the experimental procedure (FIG. 6A). While control and β-actin DNA vaccinated rats developed a sever manifestation of disease (mean maximal score 3.5±0.23 and 3.3±0.23 respectively) those treated with IP-10 encoding DNA developed significantly lower form of disease (mean maximal score 1.5±0.66, p<0.01 compared with each control group) and went into fast remission. On day 16 blood sera from these rats were analyzed for IP-10 or β-actin specific antibody titer (IgG). In accordance with the results summarized in FIGS. 6A-B, control rats with developing EAE developed a significant (p<0.05) antibody titer to IP-10 during the course of disease that was amplified (p<0.001) following DNA vaccination (FIG. 6B). EAE rats did not mount an increased antibody titer to self β-actin and this titer did not accelerate within few days after rats were subjected to β-actin encoding DNA vaccination (FIG. 6B). This further suggest that naked DNA vaccination encoding proinflammatory mediators augments a pre-existing response, that plays a function in the regulation of the autoimmune condition. These antibodies were neutralizing in vivo and could suppress EAE in adoptive transfer experiments.

Since in the Lewis rat model of MS the disease manifests a short acute form that persists for only 5-6 days, this model is not optimal for exploring the ability of IP-10 encoding DNA vaccines to treat a full blown disease. Other two commonly used models of MS in rodents are the SJL mice model for relapsing remitting MS, and the MOG p35-55 induced disease in C57/BL mice. Each of these models shares properties with human MS (116). Under the experimental conditions employed SJL mice develop a short form of disease that persisted for 5-7 days. Later on, 60-80% of the recovered mice develop a short relapse. MOG induced disease persists for about 30 days (FIG. 7). This makes this model more practical for studying a full blown disease. C57/BL mice were subjected to active induction of EAE. On day 15 these mice were separated into four groups of equally sick mice (six mice per group) and subjected to a repeated administration (3 times, days 15, 16, 17) of either IP-10 or soluble β-actin encoding DNA vaccines, empty vector (100 μg each) or PBS. Only those administered with the IP-10 encoding DNA vaccine went into a rapid remission (FIG. 7, day 23, 0.66±0.26 Vs 2.2±0.3, 2.3±0.23 and 2.5±0.3 respectively, p<0.01). At this time (day 25) sera from 3 mice per group were analyzed for the development of anti IP-10 and anti β-actin antibodies as described in with respect to FIGS. 6A-B. Mice administered with IP-10 encoding DNA vaccine developed a significantly elevated antibody titer to IP-10 (log2 Ab titer of 22±1 Vs 13±0.5 in control EAE, p<0.001, and 8±0 in naive mice). This further implies that IP-10 encoding DNA vaccination can be used as a powerful tool to generate protective autoimmunity to IP-10 and thus treat ongoing MS.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

### REFERENCES CITED

### Additional References are Cited Within in the Text

1. Alvord, E. C. J., M. W. Kies, and A. J. Suckling. 1984. Experimental Allergic Encephalomylitis: A useful Model for Multiple Sclerosis. In Progress in clinical and biological research., vol. 146. E. C. J. Alvord, M. W. Kies, and A. J. Suckling, eds. Allen R. Liss, New York, p. 1-537.

2. MacFarlin, D., and H. MacFarland. 1983. Multiple Sclerosis. N. Engl. J. Med. 307:1183-1188.

3. Karin, N., F. Szafer, D. Mitchell, D. P. Gold, and L. Steinman. 1993. Selective and nonselective stages in homing of T lymphocytes to the central nervous system during experimental allergic encephalomyelitis. J Immunol 150:4116-24.

4. Yednock, T. A., C. Cannon, L. C. Fritz, M. F. Sanchez, L. Steinman, and N. Karin. 1992. Prevention of experimental autoimmune encephalomyelitis by antibodies against alpha 4 beta 1 integrin. Nature 356:63-6.

5. Brocke, S., K. Gijbels, M. Allegretta, I. Ferber, C. Piercy, T. Blankenstein, R. Martin, U. Utz, N. Karin, D. Mitchell, and at al. 1996. Treatment of experimental encephalomyelitis with a peptide analog of myelin basic protein. Nature 379:343-6.

6. Schmied, M., H. Breitschopf, R. Gold, H. Zischler, G. Rothe, H. Wekerle, and H. Lassmann. 1993. Apoptosis of T lymphocytes in experimental autoimmune encephalomyelitis; Evidence for programmed cell death as a mechanism to control inflammation in the brain. American Journal of Pathology 143:446-451.

7. Karin, N., J. D. Mitchell, S. Brocke, N. Ling, and L. Steinman. 1994. Reversal of experimental autoimmune encephalomyelitis by as soluble peptide variant of a myelin basic protein epitope: T cell receptor antagonism and reduction of IFN-g and TNF-α production. J Exp. Med. 180:2227-2237.

8. Xiang, Z., and H. C. Ertl. 1995. Manipulation of the immune response to a plasmid-encoded viral antigen by coinoculation with plasmids expressing cytokines. Immunity 2:129-35.

9. Irvine, K. R., J. B. Rao, S. A. Rosenberg, and N. P. Restifo. 1996. Cytokine enhancement of DNA immunization leads to effective treatment of established pulmonary metastases. Journal of Immunology 156:238-45.

10. Ulmer, J. B., J. C. Sadoff, and M. A. Liu. 1996. DNA vaccines. Current opinion in immunology 8:531-536.

11. Barry, M. A., W. C. Lai, and S. A. Johnston. 1995. Protection against mycoplasma infection using expression-library immunization. Nature 377:632-5.

12. Sedegah, M., R. Hedstrom, P. Hobart, and S. L. Hoffman. 1994. Protection against malaria by immunization with plasmid DNA encoding circumsporozoite protein. Proc Natl Acad Sci U S A 91:9866-70.

13. Tang, D. C., M. DeVit, and S. A. Johnston. 1992. Genetic immunization is a simple method for eliciting an immune response. Nature 356:152-4.

14. Ulmer, J. B., J. J. Donnelly, S. E. Parker, G. H. Rhodes, P. L. Felgner, V. J. Dwarki, S. H. Gromkowski, R. R. Deck, C. M. DeWitt, A. Friedman, and at al. 1993. Heterologous protection against influenza by injection of DNA encoding a viral protein [see comments]. Science 259:1745-9.

15. Sato, Y., M. Roman, H. Tighe, D. Lee, M. Corr, M. Nguyen, G. J. Silverman, M. Lotz, D. A. Carson, and E. Raz. 1996. Immunostimulatory DNA sequences necessary for effective intradermal gene immunization. Science 273:352-357.

16. Singh, R. R., V. Kumar, F. M. Ebling, S. Southwood, A. Sette, E. E. Sercarz, and B. H. Hahn. 1995. T cell determinants from autoantibodies to DNA can upregulate autoimmunity in murine systemic lupus erythematosus. Journal of Experimental Medicine 181:2017-27.

17. Waisman, A., P. J. Ruiz, D. L. Hirschberg, A. Gelman, J. R. Oksenberg, S. Brocke, F. Mor, I. R. Cohen, and L. Steinman. 1996. Suppressive vaccination with DNA encoding a variable region gene of the T-cell receptor prevents autoimmune encephalomyelitis and activates Th2 immunity. Nature Medicine 2:899-905.

18. Bazan, J. F., K. B. Bacon, G. Hardiman, W. Wang, K. Soo, D. Rossi, D. R. Greaves, A. Ziotnik, and T. J. Schall. 1997. A new class of membrane-bound chemokine with a CX3C motif. Nature 385:640-4.

19. Pan, Y., C. Lloyd, H. Zhou, S. Dolich, J. Deeds, J. A. Gonzalo, J. Vath, M. Gosselin, J. Ma, B. Dussault, E. Woolf, G. Alperin, J. Culpepper, J. C. Gutierrez-Ramos, and D. Gearing. 1997. Neurotactin, a membrane-anchored chemokine upregulated in brain inflammation. Nature 387:611-7.

20. Ben-Baruch, A., D. F. Michiel, and J. J. Oppenheim. 1995. Signals and receptors involved in recruitment of inflammatory cells. J Biol Chem 270:11703-6.

21. Ponath, P. D., S. Qin, D. J. Ringler, I. Clark-Lewis, J. Wang, N. Kassam, H. Smith, X. Shi, J. A. Gonzalo, W. Newman, J. C. Gutierrez-Ramos, and C. R. Mackay. 1996. Cloning of the human eosinophil chemoattractant, eotaxin. Expression, receptor binding, and functional properties suggest a mechanism for the selective recruitment of eosinophils. J Clin Invest 97:604-12.

22. Carr, M. W., R. Alon, and T. A. Springer. 1996. The C-C chemokine MCP-1 differentially modulates the avidity of beta 1 and beta 2 integrins on T lymphocytes. Immunity 4:179-87.

23. Lloyd, A. R., J. J. Oppenheim, D. J. Kelvin, and D. D. Taub. 1996. Chemokines regulate T cell adherence to recombinant adhesion molecules and extracellular matrix proteins. Journal of Immunology 156:932-8.

24. Bacon, K. B., L. Flores-Romo, J. P. Aubry, T. N. Wells, and C. A. Power. 1994. Interleukin-8 and RANTES induce the adhesion of the human basophilic cell line KU-812 to human endothelial cell monolayers. Immunology 82.473-81.

25. Brown, Z., M. E. Gerritsen, W. W. Carley, R. M. Strieter, S. L. Kunkel, and J. Westwick. 1994. Chemokine gene expression and secretion by cytokine-activated human microvascular endothelial cells. Differential regulation of monocyte chemoattractant protein-i and interleukin-8 in response to interferon-gamma. American Journal of Pathology 145:913-21.

26. Carlos, T. M., and J. M. Harlan. 1994. Leukocyte-endothelial adhesion molecules. Blood 84:2068-101.

27. Jutila, M. A. 1994. Role of changes in the vascular endothelium in chronic inflammation. Clinical Transplantation 8:304-7.

28. Kim, J. S., S.C. Gautam, M. Chopp, C. Zaloga, M. L. Jones, P. A. Ward, and K. M. Welch. 1995. Expression of monocyte chemoattractant protein-i and macrophage inflammatory protein-I after focal cerebral ischemia in the rat. Journal of Neuroimmunology 56:127-34.

29. Lukacs, N. W., R. M. Strieter, V. Elner, H. L. Evanoff, M. D. Burdick, and S. L. Kunkel. 1995. Production of chemokines, interleukin-8 and monocyte chemoattractant protein-1, during monocyte: endothelial cell interactions. Blood 86:2767-73.

30. Schall, T. J., K. Bacon, R. D. Camp, J. W. Kaspari, and D. V. Goeddel. 1993. Human macrophage inflammatory protein alpha (MIP-1 alpha) and MIP-1 beta chemokines attract distinct populations of lymphocytes. Journal of Experimental Medicine 177:1821-6.

31. Vaddi, K., and R. C. Newton. 1994. Regulation of monocyte integrin expression by beta-family chemokines. Journal of Immunology 153:4721-32.

32. Yu, X., and D. T. Graves. 1995. Fibroblasts, mononuclear phagocytes, and endothelial cells express monocyte chemoattractant protein-1 (MCP-1) in inflamed human gingiva. Journal of Periodontology 66:80-8.

33. Karpus, W. J., N. W. Lukacs, B. L. McRae, R. M. Strieter, S. L. Kunkel, and S. D. Miller. 1995. An important role for the chemokine macrophage inflammatory protein-i alpha in the pathogenesis of the T cell-mediated autoimmune disease, experimental autoimmune encephalomyelitis. J. Immunol 155:5003-10.

34. Gong, J. H., L. G. Ratkay, J. D. Waterfield, and I. Clark-Lewis. 1997e. An antagonist of monocyte chemoattractant protein 1 (MCP-1) inhibits arthritis in the MRL-1pr mouse model. J Exp Med 186:131-7.

35. Berman, J. W., M. P. Guida, J. Warren, J. Amat, and C. F. Brosnan. 1996. Localization of monocyte chemoattractant peptide-1 expression in the central nervous system in experimental autoimmune encephalomyelitis and trauma in the rat. J. Immunol. 156:3017-3023.

36. Rollins, B. J. Chemokines. Blood 90, 909-928 (1997).

37. Sallusto, F., Lanzavecchia, A. & Mackay, C. R. Chemokines and chemokine receptors in T-cell priming and Th1/Th2-mediated responses. Immunol Today 19, 568-74 (1998).

38. Ward, S. G., Bacon, K. & Westwick, J. Chemokines and T lymphocytes: more than an attraction. Immunity 9, 1-11 (1998).

39. Raz, E., H. Tighe, Y. Sato, M. Corr, J. A. Dudler, M. Roman, S. L. Swain, H. L. Spiegelberg, and D. A. Carson. 1996. Preferential induction of a Th1 immune response and inhibition of specific IgE antibody formation by plasmid DNA immunization. Proc Natl Acad Sci USA 93:5141-5.

40. Godiska, R., D. Chantry, G. N. Dietsch, and P. W. Gray. 1995. Chemokine expression in murine experimental allergic encephalomyelitis. J Neuroimmunol 58: 167-76.

41. Kim, J. J., M. L. Bagarazzi, N. Trivedi, Y. Hu, K. Kazahaya, D. M. Wilson, R. Ciccarelli, M. A. Chattergoon, K. Dang, S. Mahalingam, A. A. Chalian, M. G. Agadjanyan, J. D. Boyer, B. Wang, and D. B. Weiner. 1997. Engineering of in vivo immune responses to DNA immunization via codelivery of costimulatory molecule genes. Nat Biotechnol 15:641-6.

42. Kim, J. J., V. Ayyavoo, M. L. Bagarazzi, M. A. Chattergoon, K. Dang, B. Wang, J. D. Boyer, and D. B. Weiner. 1997. In vivo engineering of a cellular immune response by coadministration of IL-12 expression vector with a DNA immunogen. J Immunol 158:816-26.

43. Fu, T. M., J. B. Ulmer, M. J. Caulfield, R. R. Deck, A. Friedman, S. Wang, X. Liu, J. J. Donnelly, and M. A. Liu. 1997. Priming of cytotoxic T lymphocytes by DNA vaccines: requirement for professional antigen presenting cells and evidence for antigen transfer from myocytes. Mol Med 3:362-71.

44. Carr, M. W., S. J. Roth, E. Luther, S. S. Rose, and T. A. Springer. 1994. Monocyte chemoattractant protein 1 acts as a T-lymphocyte chemoattractant. Proc Natl Acad Sci U S A 91:3652-6.

45. Lloyd, C. M., A. W. Minto, M. E. Dorf, A. Proudfoot, T. N. Wells, D. J. Salant, and J. C. Gutierrez-Ramos. 1997. RANTES and monocyte chemoattractant protein-i (MCP-1) play an important role in the inflammatory phase of crescentic nephritis, but only MCP-1 is involved in crescent formation and interstitial fibrosis. J Exp Med 185:1371-80.

46. Uguccioni, M., M. D'Apuzzo, M. Loetscher, B. Dewald, and M. Baggiolini. 1995. Actions of the chemotactic cytokines MCP-1, MCP-2, MCP-3, RANTES, MIP-1 alpha and MIP-1 beta on human monocytes. Eur J Immunol 25:64-8.

47. del Pozo, M. A., P. Sanchez-Mateos, M. Nieto, and F. Sanchez-Madrid. 1995. Chemokines regulate cellular polarization and adhesion receptor redistribution during lymphocyte interaction with endothelium and extracellular matrix. Involvement of cAMP signaling pathway. J Cell Biol 131:495-508.

48. Lukacs, N. W., R. M. Strieter, V. M. Elner, H. L. Evanoff, M. Burdick, and S. L. Kunkel. 1994. Intercellular adhesion molecule-1 mediates the expression of monocyte-derived MIP-1 alpha during monocyte-endothelial cell interactions. Blood 83:1174-8.

49. Weber, C., R. Alon, B. Moser, and T. A. Springer. 1996. Sequential regulation of alpha 4 beta 1 and alpha 5 beta 1 integrin avidity by CC chemokines in monocytes: implications for transendothelial chemotaxis. J Cell Biol 134:1063-73.

50. Riethmuller, G., E. P. Rieber, S. Kiefersauer, J. Prinz, P. van der Lubbe, B. Meiser, F. Breedveld, J. Eisenburg, K. Kruger, K. Deusch, and at al. 1992. From antilymphocyte serum to therapeutic monoclonal antibodies: first experiences with a chimeric CD4 antibody in the treatment of autoimmune disease. Immunol Rev 129:81-104.

51. Green, L. L., M. C. Hardy, C. E. Maynard-Currie, H. Tsuda, D. M. Louie, M. J. Mendez, H. Abderrahim, M. Noguchi, D. H. Smith, Y. Zen and at al. 1994. Antigen-specific human monoclonal antibodies from mice engineered with human Ig heavy and light chain YACs. Nat Genet 7:13-21.

52. Glabinski, A. R., M. Tani, R. M. Strieter, V. K. Tuohy, and R. M. Ransohoff. 1997. Synchronous synthesis of alpha- and beta-chemokines by cells of diverse lineage in the central nervous system of mice with relapses of chronic experimental autoimmune encephalomyelitis. Am J Pathol 150:617-30.

53. Issazadeh, S., A. Ljungdahl, B. Hojeberg, M. Mustafa, and T. Olsson. 1995. Cytokine production in the central nervous system of Lewis rats with experimental autoimmune encephalomyelitis: dynamics of mRNA expression for interleukin-10, interleukin-12, cytolysin, tumor necrosis factor alpha and tumor necrosis factor beta. J Neuroimmunol 61:205-12.

54. Kennedy, M. K., D. S. Torrance, K. S. Picha, and K. M. Mohler. 1992. Analysis of cytokine mRNA expression in the central nervous system of mice with experimental autoimmune encephalomyelitis reveals that IL-10 mRNA expression correlates with recovery. J Immunol 149:2496-505.

55. Villarroya, H., Y. Marie, J. C. Ouallet, F. Le Saux, J. L. chelingerian, and N. Baumann. 1997. Expression of TNF alpha in central neurons of Lewis rat spinal cord after EAE induction. J Neurosci Res 49:592-9.

56. Liu, J., M. W. Marino, G. Wong, D. Grail, A. Dunn, J. Bettadapura, A. J. Slavin, L. Old, and C. C. Bernard. 1998. TNF is a potent anti-inflammatory cytokine in autoimmune-mediated demyelination. Nature Medicine 4:78-83.

57. Kuroda, Y., and Y. Shimamoto. 1991. Human tumor necrosis factor-alpha augments experimental allergic encephalomyelitis in rats. J Neuroimmunol 34:159-64.

58. Powell, M. B., D. Mitchell, J. Lederman, J. Buckmeier, S. S. Zamvil, M. Graham, N. H. Ruddle, and L. Steinman. 1990. Lymphotoxin and tumor necrosis factor-alpha production by myelin basic protein-specific T cell clones correlates with encephalitogenicity. Int Immunol 2:539-44.

59. Ruddle, N. H., C. M. Bergman, K. M. McGrath, E. G. Lingenheld, M. L. Grunnet, S. J. Padula, and R. B. Clark. 1990. An antibody to lymphotoxin and tumor necrosis factor prevents transfer of experimental allergic encephalomyelitis. J Exp Med 172:1193-200.

60. Selmaj, K., C. S. Raine, and A. H. Cross. 1991. Anti-tumor necrosis factor therapy abrogates autoimmune demyelination. Ann Neurol 30:694-700.

61. Thornhill, M. H., S. M. Wellicome, D. L. Mahiouz, J. S. Lanchbury, A. U. Kyan, and D. 0. Haskard. 1991. Tumor necrosis factor combines with IL-4 or IFN-gamma to selectively enhance endothelial cell adhesiveness for T cells. The contribution of vascular cell adhesion molecule-1-dependent and independent binding mechanisms. J Immunol 146:592-8.

62. Korner, H., F. A. Lemckert, G. Chaudhri, S. Etteldorf, and J. D. Sedgwick. 1997. Tumor necrosis factor blockade in actively induced experimental autoimmune encephalomyelitis prevents clinical disease despite activated T cell infiltration to the central nervous system. Eur J Immunol 27:1973-81.

63. Korner, H., D. S. Riminton, D. H. Strickland, F. A. Lemckert, J. D. Pollard, and J. D. Sedgwick. 1997. Critical points of tumor necrosis factor action in central nervous system autoimmune inflammation defined by gene targeting. J Exp Med 186:1585-90.

64. Suen, W. E., C. M. Bergman, P. Hjelmstrom, and N. H. Ruddle. 1997. A critical role for lymphotoxin in experimental allergic encephalomyelitis. J Exp Med 186:1233-40.

65. Taupin, V., T. Renno, L. Bourbonniere, A. C. Peterson, M. Rodriguez, and T. Owens. 1997. Increased severity of experimental autoimmune encephalomyelitis, chronic macrophage/microglial reactivity, and demyelination in transgenic mice producing tumor necrosis factor-alpha in the central nervous system. Eur J Immunol 27:905-13.

66. Brenner, T., S. Brocke, F. Szafer, R. A. Sobel, J. F. Parkinson, D. H. Perez, and L. Steinman. 1997. Inhibition of nitric oxide synthase for treatment of experimental autoimmune encephalomyelitis. J Immunol 158:2940-6.

67. Frei, K., H. P. Eugster, M. Bopst, C. S. Constantinescu, E. Lavi, and A. Fontana. 1997. Tumor necrosis factor alpha and lymphotoxin alpha are not required for induction of acute experimental autoimmune encephalomyelitis. J Exp Med 185:2177-82.

68. Pan, W., W. A. Banks, M. K. Kennedy, E. G. Gutierrez, and A. J. Kastin. 1996. Differential permeability of the BBB in acute EAE: enhanced transport of TNT-alpha. Am J Physiol 271:E636-42.

69. Becher, B., V. Dodelet, V. Fedorowicz, and J. P. Antel. 1996. Soluble tumor necrosis factor receptor inhibits interleukin 12 production by stimulated human adult microglial cells in vivo. J Clin Invest 98:1539-43.

70. Leonard, J. P., K. E. Waldburger, and S. J. Goldman. 1995. Prevention of experimental autoimmune encephalomyelitis by antibodies against interleukin 12. J Exp Med 181:381-6.

71. Selmaj, K., W. Papierz, A. Glabinski, and T. Kohno. 1995. Prevention of chronic relapsing experimental autoimmune encephalomyelitis by soluble tumor necrosis factor receptor I. J Neuroimmunol 56:135-41.

72. Pette, M., K. Fujita, B. Kitze, J. N. Whitaker, E. Albert, L. Kappos, and H. Wekerle. 1990. Myelin basic protein-specific T lymphocyte lines from MS patients and healthy individuals. Neurology 40:1770-6.

73. Chen, Y., V. K. Kuchroo, J. Inobe, D. Hafler, and H. L. Weiner. 1994. Regulatory T-cell clones induced by oral tolerance: Suppression of autoimmune encephalomyelitis. Science 265:1237-1240.

74. Rapoport, M. J., A. Jaramillo, D. Zipris, A. Lazarus, D. V. Serreze, E. H. Leiter, P. Cyopick, J. S. Danska, and T. L. Delovitch. 1993. Interleukin-4 reverses T cell proliferative unresponsiveness and prevents the onset of diabetes in nonobese diabetic mice. J Exp. Med. 178:87-99.

75. Friedman, A., and H. L. Weiner. 1994. Induction of anergy or active suppression following oral tolerance is determined by antigen dosage. Proc Natl Acad Sci USA 91.6688-6692.

76. Khoury, S. J., W. W. Hancock, and H. L. Weiner. 1992. Oral tolerance to myelin basic protein and natural recovery from experimental autoimmune encephalomyelitis are associated with downregulation of inflammatory cytokines and differential upregulation of transforming growth factor beta, interleukin 4, and prostaglandin E expression in the brain. J Exp Med 176:1355-64.

77. Cash, E., A. Minty, P. Ferrara, D. Caput, D. Fradelizi, and O. Rott. 1994. Macrophage-inactivating IL-13 suppresses experimental autoimmune encephalomyelitis in rats. J Immunol 153:4258-67.

78. Saoudi, A., J. Kuhn, K. Huygen, K. Y. de, T. Velu, M. Goldman, P. Druet, and B. Bellon. 1993. TH2 activated cells prevent experimental autoimmune uveoretinitis, a TH1-dependent autoimmune disease. Eur J Immunol 23:3096-103.

79. Liblau, R. S., S. M. Singer, and H. O. McDevitt. 1994. Th1and Th2 CD4+ T-Cells in the pathogenesis of organ-specific autoimmune diseases. Immunol. Today in press.

80. Racke, M. K., J. S. Dhib, B. Cannella, P. S. Albert, C. S. Raine, and D. E. McFarlin. 1991. Prevention and treatment of chronic relapsing experimental allergic encephalomyelitis by transforming growth factor-beta 1. J Immunol 146:3012-7.

81. Racke, M. K., A. Bonomo, D. E. Scott, B. Cannella, A. Levine, C. S. Raine, E. M. Shevach, and M. Rocken. 1994. Cytokine-induced immune deviation as a therapy for inflammatory autoimmune disease. J Exp Med 180:1961-6.

82. Segal, B. M., B. K. Dwyer, and E. M. Shevach. 1998. An interleukin (IL)-10/IL-12 immunoregulatory circuit controls susceptibility to autoimmune disease. J Exp Med 187:537-46.

83. Steinman, L. 1995. Escape from “horror autotoxicus”: pathogenesis and treatment of autoimmune disease. Cell 80:7-10.

84. Matzinger, P. 1994. Tolerance, danger, and the extended family. Annu Rev Immunol 12:991-1045.

85. Janeway, C. A., Jr. 1992. The immune system evolved to discriminate infectious nonself from noninfectious self. Immunol Today 13:11-6.

86. Cyster, J. G., S. B. Hartley, and C. C. Goodnow. 1994. Competition for follicular niches excludes self-reactive cells from the recirculating B-cell repertoire. Nature 371:389-95. 87. Barnes, D. A. et al. Polyclonal antibody directed against human RANTES ameliorates disease in the Lewis rat adjuvant-induced arthritis model. J Clin Invest 101, 2910-9 (1998).

88. Hemmi, H. et al. A Toll-like receptor recognizes bacterial DNA. Nature 408, 740-5 (2000).

89. Wen-Ming, C. et al. DNA-PKcs Is Required for Activation of Innate Immunity by Immunostimulatory DNA. Cell 103, 909-918 (2000).

90. Wildbaum, G. & Karin, N. Augmentation of natural immunity to a pro-inflammatory cytokine (TNF-alpha) by targeted DNA vaccine confers long-lasting resistance to experimental autoimmune encephalomyelitis. Gene Ther 6, 1128-38 (1999).

91. Wildbaum, G., Westermann, J., Maor, G. & Karin, N. A targeted DNA vaccine encoding fas ligand defines its dual role in the regulation of experimental autoimmune encephalomyelitis [In Process Citation]. J Clin Invest 106, 671-9 (2000).

92. Wildbaum, G., Youssef, S. & Karin, N. A targeted DNA vaccine augments the natural immune response to self TNF-a and suppresses adjuvant arthritis. J Immunol. 165, 5860-58666 (2000).

93. Wildbaum, G., Youssef, S. & Karin, N. A Targeted DNA Vaccine Augments the Natural Immune Response to Self TNF-alpha and Suppresses Ongoing Adjuvant Arthritis. J Immunol 165, 5860-5866 (2000).

94. Youssef, S. et al. Long lasting protective immunity to experimental autoimmune encephalomyelitis following vaccination with naked DNA encoding C-C chemokines. J. Immunol 161, 3870-3879 (1998).

95. Youssef, S., Wildbaum, G. & Karin, N. Prevention of Experimental Autoimmune Encephalomyelitis by MIP-1 alpha and MCP-1 Naked DNA Vaccines. J Autoimmun 13, 21-29 (1999).

96. Youssef, S. et al. C-C chemokine-encoding DNA vaccines enhance breakdown of tolerance to their gene products and treat ongoing adjuvant arthritis. J Clin Invest 106, 361-371 (2000).

97. Cole, K. E. et al. Interferon-inducible T cell alpha chemoattractant (1-TAC): a novel non-ELR CXC chemokine with potent activity on activated T cells through selective high affinity binding to CXCR3. Journal of Experimental Medicine 187,2009-21(1998).

98. Sallusto, F., Lenig, D., Mackay, C. R. & Lanzavecchia, A. Flexible programs of chemokine receptor expression on human polarized T helper 1 and 2 lymphocytes. Journal of Experimental Medicine 187, 875-83 (1998).

99. Xie, H., Lim, Y. C., Luscinskas, F. W. & Lichtman, A.H. Acquisition of selectin binding and peripheral homing properties by CD4(+) and CD8(+) T cells. J Exp Med 189, 1765-76 (1999).

100. Taub, D. D., Longo, D. L. & Murphy, W. J. Human interferon-inducible protein-10 induces mononuclear cell infiltration in mice and promotes the migration of human T lymphocytes into the peripheral tissues and human peripheral blood lymphocytes-SCID mice. Blood 87, 1423-31 (1996).

101. Balashov, K. E., Rottman, J. B., Weiner, H. L. & Hancock, W. W. CCR5(+) and CXCR3(+) T cells are increased in multiple sclerosis and their ligands MIP-1alpha and IP-10 are expressed in demyelinating brain lesions. Proceedings of the National Academy of Sciences of the United States of America 96, 6873-8 (1999).

102. Bradley, L. M. et al. Islet-specific Th1, but not Th2, cells secrete multiple chemokines and promote rapid induction of autoimmune diabetes. Journal of Immunology 162, 2511-20 (1999).

103. Ransohoff, R. M. et al. Astrocyte expression of mRNA encoding cytokines IP-10 and JE/MCP-1 in experimental autoimmune encephalomyelitis. FASEB Journal 7, 592-600 (1993).

104. Sorensen, T. L. et al. Expression of specific chemokines and chemokine receptors in the central nervous system of multiple sclerosis patients. Journal of Clinical Investigation 103, 807-15 (1999).

105. Wildbaum, G., Youssef, S., Grabie, N. & Karin, N. Prevention of experimental autoimmune encephalomyelitis by antibodies to interferon gamma inducing factor. J. Immunol 161, 6368-6374 (1998).

106. Boyer, J. D. et al. Protection of chimpanzees from high-dose heterologous HIV-1 challenge by DNA vaccination. Nat Med 3, 526-32 (1997).

107. Kim, J. J. et al. In vivo engineering of a cellular immune response by coadministration of IL-12 expression vector with a DNA immunogen. J Immunol 158, 816-26 (1997).

108. Kim, J. J. et al. Engineering of in vivo immune responses to DNA immunization via codelivery of costimulatory molecule genes. Nat Biotechnol 15, 641-6 (1997).

109. Raz, E. et al. Systemic immunological effects of cytokine genes injected into skeletal muscle. Proc Natl Acad Sci U S A 90, 4523-7 (1993).

110. Tascon, R. E. et al. Vaccination against tuberculosis by DNA injection. Nat Med 2, 888-92 (1996).

111. Ruiz, P. J. et al. Suppressive immunization with DNA encoding a self-peptide prevents autoimmune disease: modulation of T cell costimulation. J Immunol 162, 3336-41 (1999).

112. Hogger, P., Dreier, J., Droste, A., Buck, F. & Sorg, C. Identification of the integral membrane protein RM3/1 on human monocytes as a glucocorticoid-inducible member of the scavenger receptor cysteine-rich family (CD163). Journal of Immunology 161, 1883-90 (1998).

113. Balashov, K. E., J. B. Rottman, H. L. Weiner, and W. W. Hancock. 1999. CCR5(+) and CXCR3(+) T cells are increased in multiple sclerosis and their ligands MIP-1alpha and IP-10 are expressed in demyelinating brain lesions. Proceedings of the National Academy of Sciences of the United States of America 96:6873.

114. Narumi, S., Y. Tominaga, M. Tamaru, S. Shimai, H. Okumura, K. Nishioji, Y. Itoh, and T. Okanoue. 1997. Expression of IFN-inducible protein-10 in chronic hepatitis. Journal of Immunology 158:5536.

115. Qin, S., J. B. Rottman, P. Myers, N. Kassam, M. Weinblatt, M. Loetscher, A. E. Koch, B. Moser, and C. R. Mackay. 1998. The chemokine receptors CXCR3 and CCR5 mark subsets of T cells associated with certain inflammatory reactions. Journal of Clinical Investigation 101: 746.

116. Wekerle, H. 1999. Remembering MOG: autoantibody mediated demyelination in multiple sclerosis? [news; comment]. Nature Medicine 5:153.

What is claimed is:

1. A method of breaking-down an immunological tolerance to interferon gamma-inducible protein 10 in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to break-down the immunological tolerance to interferon gamma-inducible protein 10.

2. The method of claim 1, wherein expressing within the subject said amount of interferon gamma-inducible protein 10 is by generating in, or introducing into, the subject cells expressing recombinant interferon gamma-inducible protein 10, or an immunological portion thereof.

3. The method of claim 1, wherein generating in the subject cells expressing recombinant interferon gamma-inducible protein 10, or an immunological portion thereof, is by vaccinating the subject with an expression construct encoding interferon gamma-inducible protein 10, or said immunological portion thereof.

4. A method of generating self specific immunity to interferon gamma-inducible protein 10 in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to generate self specific immunity to interferon gamma-inducible protein 10.

5. The method of claim 4, wherein expressing within the subject said amount of interferon gamma-inducible protein 10 is by generating in, or introducing into, the subject cells expressing recombinant interferon gamma-inducible protein 10, or an immunological portion thereof.

6. The method of claim 4, wherein generating in the subject cells expressing recombinant interferon gamma-inducible protein 10, or an immunological portion thereof, is by vaccinating the subject with an expression construct encoding interferon gamma-inducible protein 10, or said immunological portion thereof.

7. A method of preventing or treating an autoimmune disease in which activity of interferon gamma-inducible protein 10 is pivotal in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to treat or prevent the autoimmune disease.

8. The method of claim 7, wherein expressing within the subject said amount of interferon gamma-inducible protein 10 is by generating in, or introducing into, the subject cells expressing recombinant interferon gamma-inducible protein 10, or an immunological portion thereof.

9. The method of claim 7, wherein generating in the subject cells expressing recombinant interferon gamma-inducible protein 10, or an immunological portion thereof, is by vaccinating the subject with an expression construct encoding interferon gamma-inducible protein 10, or said immunological portion thereof.

10. A method of restricting a polarization of myelin basic protein specific T cells into Th2 cells in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to restrict a polarization of myelin basic protein specific T cells into Th2 cells.

11. The method of claim 10, wherein expressing within the subject said amount of interferon gamma-inducible protein 10 is by generating in, or introducing into, the subject cells expressing recombinant interferon gamma-inducible protein 10, or an immunological portion thereof.

12. The method of claim 10, wherein generating in the subject cells expressing recombinant interferon gamma-inducible protein 10, or an immunological portion thereof, is by vaccinating the subject with an expression construct encoding interferon gamma-inducible protein 10, or said immunological portion thereof.

13. A method of inducing protective immunity against multiple sclerosis in a subject, the method comprising administering to, or expressing within, the subject an amount of interferon gamma-inducible protein 10, or an immunological portion thereof, sufficient to elicit sufficient anti-interferon gamma-inducible protein 10 antibodies, so as to induce protective immunity against multiple sclerosis in the subject.

14. The method of claim 13, wherein expressing within the subject said amount of interferon gamma-inducible protein 10 is by generating in, or introducing into, the subject cells expressing recombinant interferon gamma-inducible protein 10, or an immunological portion thereof.

15. The method of claim 13, wherein generating in the subject cells expressing recombinant interferon gamma-inducible protein 10, or an immunological portion thereof, is by vaccinating the subject with an expression construct encoding interferon gamma-inducible protein 10, or said immunological portion thereof.

16. A method of preventing or treating multiple sclerosis in a subject, the method comprising administering to, or expressing within, the subject an amount of interferon gamma-inducible protein 10, or an immunological portion thereof, sufficient to elicit sufficient anti-interferon gamma-inducible protein 10 antibodies so as to treat or prevent multiple sclerosis.

17. The method of claim 16, wherein expressing within the subject said amount of interferon gamma-inducible protein 10 is by generating in, or introducing into, the subject cells expressing recombinant interferon gamma-inducible protein 10, or an immunological portion thereof.

18. The method of claim 16, wherein generating in the subject cells expressing recombinant interferon gamma-inducible protein 10, or an immunological portion thereof, is by vaccinating the subject with an expression construct encoding interferon gamma-inducible protein 10, or said immunological portion thereof.

19. A pharmaceutical composition for breaking-down an immunological tolerance to interferon gamma-inducible protein 10 in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding said interferon gamma-inducible protein 10, or said immunological portion thereof, sufficient to elicit sufficient anti-interferon gamma-inducible protein 10 antibodies so as to break-down the immunological tolerance to interferon gamma-inducible protein 10.

20. The pharmaceutical composition of claim 19, packaged and identified for treatment of a disease or condition in which interferon gamma-inducible protein 10 plays a pivotal role.

21. A pharmaceutical composition for generating self specific immunity to interferon gamma-inducible protein 10 in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding said interferon gamma-inducible protein 10, or said immunological portion thereof, sufficient to elicit sufficient anti-interferon gamma-inducible protein 10 antibodies so as to generate self specific immunity to interferon gamma-inducible protein 10.

22. The pharmaceutical composition of claim 21, packaged and identified for treatment of a disease or condition in which interferon gamma-inducible protein 10 plays a pivotal role.

23. A pharmaceutical composition for preventing or treating an autoimmune disease in which activity of interferon gamma-inducible protein 10 is pivotal in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding said interferon gamma-inducible protein 10, or said immunological portion thereof, sufficient to elicit sufficient anti-interferon gamma-inducible protein 10 antibodies so as to treat or prevent the autoimmune disease.

24. The pharmaceutical composition of claim 23, packaged and identified for treatment of a disease or condition in which interferon gamma-inducible protein 10 plays a pivotal role.

25. A pharmaceutical composition for restricting a polarization of myelin basic protein specific T cells into Th2 cells in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding said interferon gamma-inducible protein 10, or said immunological portion thereof, sufficient to elicit sufficient anti-interferon gamma-inducible protein 10 antibodies so as to restrict a polarization of myelin basic protein specific T cells into Th2 cells.

26. The pharmaceutical composition of claim 25, packaged and identified for treatment of a disease or condition in which interferon gamma-inducible protein 10 plays a pivotal role.

27. A pharmaceutical composition for inducing protective immunity against multiple sclerosis in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding said interferon gamma-inducible protein 10, or said immunological portion thereof, sufficient to elicit sufficient anti-interferon gamma-inducible protein 10 antibodies, so as to induce protective immunity against multiple sclerosis in the subject.

28. The pharmaceutical composition of claim 27, packaged and identified for treatment of a disease or condition in which interferon gamma-inducible protein 10 plays a pivotal role.

30. A pharmaceutical composition for preventing or treating multiple sclerosis in a subject, the pharmaceutical composition comprising, a pharmaceutically acceptable carrier approved for medical or veterinary administration and, as an active ingredient, an amount of interferon gamma-inducible protein 10, or an immunological portion thereof, or of an expression construct encoding said interferon gamma-inducible protein 10, or said immunological portion thereof, sufficient to elicit sufficient anti-interferon gamma-inducible protein 10 antibodies so as to treat or prevent multiple sclerosis.

31. The pharmaceutical composition of claim 30, packaged and identified for treatment of a disease or condition in which interferon gamma-inducible protein 10 plays a pivotal role.

32. A method of breaking-down an immunological tolerance to interferon gamma-inducible protein 10 in a subject, the method comprising administering to the subject anti-interferon gamma-inducible protein 10 antibodies in an amount sufficient to break-down the immunological tolerance to interferon gamma-inducible protein 10.

33. A method of generating specific immunity to interferon gamma-inducible protein 10 in a subject, the method comprising administering to the subject anti-interferon gamma-inducible protein 10 antibodies in an amount sufficient to generate self specific immunity to interferon gamma-inducible protein 10.

34. A method of preventing or treating an autoimmune disease in which activity of interferon gamma-inducible protein 10 is pivotal in a subject, the method comprising administering to the subject anti-interferon gamma-inducible protein 10 antibodies in an amount sufficient to treat or prevent the autoimmune disease.

35. A method of restricting a polarization of myelin basic protein specific T cells into Th2 cells in a subject, the method comprising administering to the subject anti-interferon gamma-inducible protein 10 antibodies in an amount sufficient to restrict a polarization of myelin basic protein specific T cells into Th2 cells.

36. A method of inducing protective immunity against multiple sclerosis in a subject, the method comprising administering to the subject anti-interferon gamma-inducible protein 10 antibodies in an amount sufficient to induce protective immunity against multiple sclerosis in the subject.

37. A method of preventing or treating multiple sclerosis in a subject, the method comprising administering to the subject anti-interferon gamma-inducible protein 10 antibodies in an amount sufficient to treat or prevent multiple sclerosis.

38. A pharmaceutical composition for breaking-down an immunological tolerance to interferon gamma-inducible protein 10 in a subject, the pharmaceutical composition comprising, a pharmaceutically acceptable carrier approved for medical or veterinary administration and, as an active ingredient, anti-interferon gamma-inducible protein 10 antibodies in an amount sufficient to break-down the immunological tolerance to interferon gamma-inducible protein 10.

39. The pharmaceutical composition of claim 38, packaged and identified for treatment of a disease or condition in which interferon gamma-inducible protein 10 plays a pivotal role.

40. A pharmaceutical composition for generating self specific immunity to interferon gamma-inducible protein 10 in a subject, the pharmaceutical composition comprising, a pharmaceutically acceptable carrier approved for medical or veterinary administration and, as an active ingredient, anti-interferon gamma-inducible protein 10 antibodies in an amount sufficient to generate self specific immunity to interferon gamma-inducible protein 10.

41. The pharmaceutical composition of claim 40, packaged and identified for treatment of a disease or condition in which interferon gamma-inducible protein 10 plays a pivotal role.

42. A pharmaceutical composition for preventing or treating an autoimmune disease in which activity of interferon gamma-inducible protein 10 is pivotal in a subject, the pharmaceutical composition comprising, a pharmaceutically acceptable carrier approved for medical or veterinary administration and, as an active ingredient, anti-interferon gamma-inducible protein 10 antibodies in an amount sufficient to treat or prevent the autoimmune disease.

43. The pharmaceutical composition of claim 42, packaged and identified for treatment of a disease or condition in which interferon gamma-inducible protein 10 plays a pivotal role.

44. A pharmaceutical composition for restricting a polarization of myelin basic protein specific T cells into Th2 cells in a subject, the pharmaceutical composition comprising, a pharmaceutically acceptable carrier approved for medical or veterinary administration and, as an active ingredient, anti-interferon gamma-inducible protein 10 antibodies in an amount sufficient to restrict a polarization of myelin basic protein specific T cells into Th2 cells.

45. The pharmaceutical composition of claim 44, packaged and identified for treatment of a disease or condition in which interferon gamma-inducible protein 10 plays a pivotal role.

46. A pharmaceutical composition for inducing protective immunity against multiple sclerosis in a subject, the pharmaceutical composition comprising, a pharmaceutically acceptable carrier approved for medical or veterinary administration and, as an active ingredient, anti-interferon gamma-inducible protein 10 antibodies in an amount sufficient to induce protective immunity against multiple sclerosis in the subject.

47. The pharmaceutical composition of claim 46, packaged and identified for treatment of a disease or condition in which interferon gamma-inducible protein 10 plays a pivotal role.

48. A pharmaceutical composition for preventing or treating multiple sclerosis in a subject, the pharmaceutical composition comprising, a pharmaceutically acceptable carrier approved for medical or veterinary administration and, as an active ingredient, anti-interferon gamma-inducible protein 10 antibodies in an amount sufficient to treat or prevent multiple sclerosis.

49. The pharmaceutical composition of claim 48, packaged and identified for treatment of a disease or condition in which interferon gamma-inducible protein 10 plays a pivotal role.

50. A method of breaking-down an immunological tolerance to interferon gamma-inducible protein 10 in a subject, the method comprising directly or indirectly introducing anti-interferon gamma-inducible protein 10 antibodies to the subject in an amount sufficient to break-down the immunological tolerance to interferon gamma-inducible protein 10.

51. A method of generating specific immunity to interferon gamma-inducible protein 10 in a subject, the method comprising directly or indirectly introducing anti-interferon gamma-inducible protein 10 antibodies to the subject in an amount sufficient to generate self specific immunity to interferon gamma-inducible protein 10.

52. A method of preventing or treating an autoimmune disease in which activity of interferon gamma-inducible protein 10 is pivotal in a subject, the method comprising directly or indirectly introducing anti-interferon gamma-inducible protein 10 antibodies to the subject in an amount sufficient to treat or prevent the autoimmune disease.

53. A method of restricting a polarization of myelin basic protein specific T cells into Th2 cells in a subject, the method comprising directly or indirectly introducing anti-interferon gamma-inducible protein 10 antibodies to the subject in an amount sufficient to restrict a polarization of myelin basic protein specific T cells into Th2 cells.

54. A method of inducing protective immunity against multiple sclerosis in a subject, the method comprising directly or indirectly introducing anti-interferon gamma-inducible protein 10 antibodies to the subject in an amount sufficient to induce protective immunity against multiple sclerosis in the subject.

55. A method of preventing or treating multiple sclerosis in a subject, the method comprising directly or indirectly introducing anti-interferon gamma-inducible protein 10 antibodies to the subject in an amount sufficient to treat or prevent multiple sclerosis.


SEQUENCE LISTING (3 sequences)

SEQ ID NO: 1 - protein, 17 aa, Artificial Sequence
  synthetic peptide
YGSLPQKSQRSQDENPV

SEQ ID NO: 2 - DNA, 24 bp, Artificial Sequence
  synthetic oligonucleotide
catgaacccaagtgctgctgtcgt

SEQ ID NO: 3 - DNA, 24 bp, Artificial Sequence
  synthetic oligonucleotide
ttacggagctcttttagaccttct